(19) United States
(12) Patent Application Publication
(10) Pub. No.: US 2018/0055946 A1
(43) Pub. Date: Mar. 1, 2018

(54) VERSATILE PEPTIDE-BASED MULTI-ARM LINKERS FOR CONSTRUCTING PHARMACEUTICAL MOLECULES

(71) Applicant: Immunwork Inc., Taipei City (TW)

(72) Inventors: Tse-Wen CHANG, Taipei City (TW); Hsing-Mao CHU, Taipei City (TW)

(73) Assignee: Immunwork Inc., Taipei City (TW)

(21) Appl. No.: 15/693,503

(22) Filed: Sep. 1, 2017

Related U.S. Application Data
(60) Provisional application No. 62/382,277, filed on Sep. 1, 2016.

(51) Int. Cl. A61K 47/65; A61K 47/60; C07K 5/103

(52) U.S. Cl. CPC A61K 47/65; C07K 5/1013; A61K 47/60

(57) ABSTRACT

Disclosed herein are linker units comprising a center core, a plurality of linking arms, and optionally, a coupling arm. According to the embodiments of the present disclosure, the present linker units further comprises a targeting element and an effector element. Also disclosed herein are methods for treating various diseases using such linker units.

### CROSS-REFERENCE TO RELATED APPLICATIONS

This application relates to and claims the benefit of U.S. Provisional Application No. 62/382,277, filed Sep. 01, 2016; the content of which is incorporated herein by reference in its entirety.

### BACKGROUND OF THE INVENTION

### 1. Field of the Invention

The present disclosure relates to the field of pharmaceuticals; more particularly, to multi-functional molecular constructs, e.g., those having targeting and effector elements for delivering the effector (e.g., therapeutic drug) to targeted sites.

### 2. Description of the Related Art

The continual advancement of a broad array of methodologies for screening and selecting monoclonal antibodies (mAbs) for targeted antigens has helped the development of a good number of therapeutic antibodies for many diseases that were regarded as untreatable just a few years ago. According to Therapeutic Antibody Database, more than 3600 antibodies have been studied or are being planned for studies in human clinical trials, and approximately 100 antibodies have been approved by governmental drug regulatory agencies for clinical uses. The large amount of data on the therapeutic effects of antibodies has provided information concerning the pharmacological mechanisms how antibodies act as therapeutics.

One major pharmacologic mechanism for antibodies acting as therapeutics is that, antibodies can neutralize or trap disease-causing mediators, which may be cytokines or immune components present in the blood circulation, interstitial space, or in the lymph nodes. The neutralizing activity inhibits the interaction of the disease-causing mediators with their receptors. It should be noted that fusion proteins of the soluble receptors or the extracellular portions of receptors of cytokines and the Fc portion of IgG, which act by neutralizing the cytokines or immune factors in a similar fashion as neutralizing antibodies, have also been developed as therapeutic agents.

Several therapeutic antibodies that have been approved for clinical applications or subjected to clinical developments mediate their pharmacologic effects by binding to receptors, thereby blocking the interaction of the receptors with their ligands. For those antibody drugs, Fc-mediated mechanisms, such as antibody-dependent cellular cytotoxicity (ADCC) and complement-mediated cytolysis (CMC), are not the intended mechanisms for the antibodies.

Some therapeutic antibodies bind to certain surface antigens on target cells and render Fc-mediated functions and other mechanisms on the target cells. The most important Fc-mediated mechanisms are antibody-dependent cellular cytotoxicity (ADCC) and complement-mediated cytolysis (CMC), which both will cause the lysis of the antibody-bound target cells. Some antibodies binding to certain cell surface antigens can induce apoptosis of the bound target cells.

The concept and methodology for preparing antibodies with dual specificities germinated more than three decades ago. In recent year, the advancement in recombinant antibody engineering methodologies and the drive to develop improved medicine has stimulated the development bi-specific antibodies adopting a large variety of structural configurations.

For example, the bi-valent or multivalent antibodies may contain two or more antigen-binding sites. A number of methods have been reported for preparing multivalent antibodies by covalently linking three or four Fab fragments via a connecting structure. For example, antibodies have been engineered to express tandem three or four Fab repeats.

Several methods for producing multivalent antibodies by employing synthetic crosslinkers to associate, chemically, different antibodies or binding fragments have been disclosed. One approach involves chemically cross-linking three, four, and more separately Fab fragments using different linkers. Another method to produce a construct with multiple Fabs that are assembled to one-dimensional DNA scaffold was provided. Those various multivalent Ab constructs designed for binding to target molecules differ among one another in size, half-lives, flexibility in conformation, and ability to modulate the immune system. In view of the foregoing, several reports have been made for preparing molecular constructs with a fixed number of effector elements or with two or more different kinds of functional elements (e.g., at least one targeting element and at least one effector element). However, it is often difficult to build a molecular construct with a particular combination of the targeting and effector elements either using chemical synthesis or recombinant technology. Accordingly, there exists a need in the related art to provide novel molecular platforms to build a more versatile molecule suitable for covering applications in a wide range of diseases.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a linker unit comprising a center core, a plurality of linking arms, and optionally a coupling arm having an azide, an alkyne, a tetrazine, a cyclooctene, or a cyclooctyne group at its free terminus.

According to some embodiments of the present disclosure, the center core comprises,

(1) 2 to 15 lysine (K) residues;

(2) one or more conjugating sequences, disposed at the N- or C-terminus of the center core or between two consecutive K residues of the 2 to 15 K residues, wherein each of the conjugating sequences independently comprises a conjugating amino acid residue that is a cysteine (C) residue or an amino acid residue having an azide or an alkyne group, wherein when the conjugating amino acid residue is the C residue, then the thiol group of the C residue is linked with the coupling arm; and

(3) optionally, one or more filler sequences, disposed between two consecutive K residues of the 2 to 15 K residues, wherein each of the filler sequences independently comprises two or more amino acid residues other than the conjugating amino acid residue, and at least one of the filler sequences is devoid of glycine (G), serine (S), or a combination thereof.

Preferably, the conjugating amino acid residue is not disposed at the N- or C-terminus of the center core.

According to certain embodiments of the present disclosure, the center core comprises,

(1) 2 to 15 lysine (K) residues;

(2) one or more conjugating sequences, disposed between two consecutive K residues of the 2 to 15 K residues, wherein each of the conjugating sequences independently comprises a conjugating amino acid residue that is a cysteine (C) residue or an amino acid residue having an azide or an alkyne group, wherein when the conjugating amino acid residue is the C residue, then the thiol group of the C residue is linked with the coupling arm; and

(3) optionally, one or more filler sequences, disposed between two consecutive K residues of the 2 to 15 K residues, wherein each of the filler sequences, independently, (a) comprises two or more amino acid residues other than the conjugating amino acid residue, or (b) is a PEGylated amino acid having 2 to 12 repeats of ethylene glycol (EG) unit.

Structurally, the plurality of linking arms are respectively linked to the K residues of the center core, wherein each of the plurality of linking arms has a N-hydroxysuccinimidyl (NHS), the azide, the alkyne, the tetrazine, the cyclooctene, or the cyclooctyne group at its free terminus. In the case when the free terminus of the linking arm is the azide, the alkyne, or the cyclooctyne group, then the conjugating amino acid residue is the C residue, and the free terminus of the coupling arm is the tetrazine or the cyclooctene group. In the case when the free terminus of the linking arm is the tetrazine group or cyclooctene group, then the conjugating amino acid residue is the C residue or the amino acid residue having the azide or the alkyne group and the free terminus of the coupling arm is the azide, the alkyne, or the cyclooctyne group.

According to some embodiments of the present disclosure, each of the linking arms is a PEG chain having 2-20 repeats of EG units. According to other embodiments of the present disclosure, each of the linking arms is a PEG chain having 2-20 repeats of EG units with a disulfide linkage at the free terminus thereof.

Regarding the coupling arm, it is a PEG chain having 2-12 repeats of EG units.

Optionally, the present linker unit may further comprise a plurality of connecting arms that are respectively linked to the plurality of linking arms via copper catalyzed azide-alkyne cycloaddition (CuAAC) reaction, strained-promoted azide-alkyne click chemistry (SPAAC) reaction, or inverse electron demand Diels-Alder (iEDDA) reaction, wherein each of the plurality of connecting arms has a maleimide or the NHS group at its free terminus. Similar to the linking arms, each of the connecting arms is a PEG chain having 2-20 repeats of EG units or is a PEG chain having 2-20 repeats of EG units with a disulfide linkage at the terminus that is not linked with the linking arm.

According to various embodiments of the present disclosure, the linker unit further comprises a plurality of first elements that are respectively linked to the plurality of linking arms or connecting arm via forming an amide bound therebetween, or via thiol-maleimide reaction, CuAAC reaction, SPAAC reaction, or iEDDA reaction.

Depending on desired purposes, the present linker unit may further comprise a second element that is linked to the center core via (1) CuAAC reaction occurred between the azide or the alkyne group and the second element; (2) SPAAC reaction occurred between the azide or cyclooctyne group and the second element; or (3) iEDDA reaction occurred between the cyclooctene group or tetrazine group and the second element.

Optionally, the present linker unit may further comprise a third element, in which the plurality of first elements are respectively linked to the plurality of linking arms via forming the amide bound therebetween, the second element is linked to the azide or alkyne group via CuAAC or SPAAC reaction, and the third element is linked to the coupling arm linked with the C residue via iEDDA reaction.

In general, the amino acid residue having the azide group is L-azidohomoalanine (AHA), 4-azido-L-phenylalanine, 4-azido-D-phenylalanine, 3-azido-L-alanine, 3-azido-D-alanine, 4-azido-L-homoalanine, 4-azido-D-homoalanine, 5-azido-L-ornithine, 5-azido-d-ornithine, 6-azido-L-lysine, or 6-azido-D-lysine. The amino acid residue having the alkyne group is L-homopropargylglycine (L-HPG), D-homopropargylglycine (D-HPG), or beta-homopropargylglycine (β-HPG). The cyclooctene group is trans-cyclooctene (TCO); and the cyclooctyne group is dibenzocyclooctyne (DBCO), difluorinated cyclooctyne(DIFO), bicyclononyne (BCN), or dibenzocyclooctyne (DICO). The tetrazine group is 1,2,3,4-tetrazine, 1,2,3,5-tetrazine or 1,2,4,5-tetrazine, or derivatives thereof.

The second aspect of the present disclosure is directed to a molecular construct comprising two linker units coupling to each other either directly or indirectly, in which the core of one linker unit is configured to be linked with at least one targeting element while the core of the other linker unit is configured to be linked with at least one effector element.

Structurally, the molecular construct comprises a first linker unit and a second linker unit. The first linker unit comprises a first center core and one or more linking arms (hereinafter, the first linking arms) and optionally a coupling arm (hereinafter, the first coupling arms) that are respectively linked to the first center core; the second linker unit comprises a second center core and one or more linking arms (hereinafter, the second linking arms) and optionally a coupling arm (hereinafter, the second coupling arm) that are respectively linked to the second center core. The first and second linker units are coupled to each other via iEDDA, SPAAC, or CuAAC reaction occurred between any of the followings: the first and second center cores, the first coupling arm and the second center core, the first and second coupling arms, or the first center core and the second coupling arm. For example, one of the first and second coupling arms may have an azide group at the free-terminus thereof, and the other of the first and second coupling arms may have an alkyne or a cyclooctyne group at the free-terminus thereof; in this case, the first and second linker units are coupled to each other via CuAAC reaction or SPAAC reaction occurred between the first and second coupling arms. Alternatively, the one of the first and second coupling arms may have a tetrazine group at the free-terminus thereof, and the other of the first and second coupling arms may have a cyclooctene group at the free-terminus thereof; thus, the first and second linker units are coupled to each other via iEDDA reaction occurred between the first and second coupling arms.

Optionally, the first and second linker units may further comprise a first and a second connecting arms that are respectively linked to the first and second linking arms. Each of the plurality of connecting arms has a maleimide or the NHS group at its free terminus.

According to some embodiments of the present disclosure, the present molecular construct further comprises a first and a second elements, which are respectively linked to the first and second linking arms or the first and second connecting arms via forming an amide bound therebetween, or via thiol-maleimide reaction, CuAAC reaction, SPAAC reaction, or iEDDA reaction.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings briefly discussed below.

FIG. 1A to FIG. 1Q are schematic diagrams illustrating linker units according to certain embodiments of the present disclosure.

FIG. 2A to FIG. 2D are schematic diagrams illustrating T-E molecular constructs according to some embodiments of the present disclosure.

FIG. 3 is a schematic diagram that illustrates libraries for constructing molecular constructs according to some embodiments of the present disclosure.

FIG. 4A and FIG. 4B are schematic diagrams that illustrate molecular constructs according to some embodiments of the present disclosure.

FIG. 5 is a schematic diagram that illustrates a molecular construct according to some embodiments of the present disclosure.

FIG. 6A and FIG. 6B are schematic diagrams illustrating molecular constructs according to various embodiments of the present disclosure.

FIG. 7 is the data of MALDI-TOF analysis according to Example 1 of the present disclosure.

FIG. 8 is the data of MALDI-TOF analysis according to Example 1 of the present disclosure.

FIG. 9 is the data of MALDI-TOF analysis according to Example 1 of the present disclosure.

FIGS. 10A and 10B are the data of HPLC and MALDI-TOF analysis that depict the center core comprising a coupling arm with a methyltetrazine group according to Example 2 of the present disclosure.

FIGS. 11A and 11B are the data of HPLC and MALDI-TOF analysis that depict the center core comprising a coupling arm and two linking arms, in which the coupling arm has a methyltetrazine group at its free terminus, and each of the linking arms has a maleimide at its free terminus according to Example 3 of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art.

Unless otherwise required by context, it will be understood that singular terms shall include plural forms of the same and plural terms shall include the singular. Specifically, as used herein and in the claims, the singular forms “a” and “an” include the plural reference unless the context clearly indicated otherwise. Also, as used herein and in the claims, the terms “at least one” and “one or more” have the same meaning and include one, two, three, or more. Furthermore, the phrases “at least one of A, B, and C”, “at least one of A, B, or C” and “at least one of A, B and/or C,” as use throughout this specification and the appended claims, are intended to cover A alone, B alone, C alone, A and B together, B and C together, A and C together, as well as A, B, and C together.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

This present disclosure pertains generally to molecular constructs, in which each molecular construct comprises a targeting element (T) and an effector element (E), and these molecular constructs are sometimes referred to as “T-E molecules”, “T-E pharmaceuticals” or “T-E drugs” in this document.

As used herein, the term “targeting element” refers to the portion of a molecular construct that directly or indirectly binds to a target of interest (e.g., a receptor on a cell surface or a protein in a tissue) thereby facilitates the transportation of the present molecular construct into the interested target. In some example, the targeting element may direct the molecular construct to the proximity of the target cell. In other cases, the targeting element specifically binds to a molecule present on the target cell surface or to a second molecule that specifically binds a molecule present on the cell surface. In some cases, the targeting element may be internalized along with the present molecular construct once it is bound to the interested target, hence is relocated into the cytosol of the target cell. A targeting element may be an antibody or a ligand for a cell surface receptor, or it may be a molecule that binds such antibody or ligand, thereby indirectly targeting the present molecular construct to the target site (e.g., the surface of the cell of choice). The localization of the effector (therapeutic agent) in the diseased site will be enhanced or favored with the present molecular constructs as compared to the therapeutic without a targeting function. The localization is a matter of degree or relative proportion; it is not meant for absolute or total localization of the effector to the diseased site.

According to the present invention, the term “effector element” refers to the portion of a molecular construct that elicits a biological activity (e.g., inducing immune responses, exerting cytotoxic effects and the like) or other functional activity (e.g., recruiting other hapten tagged therapeutic molecules), once the molecular construct is directed to its target site. The “effect” can be therapeutic or diagnostic. The effector elements encompass those that bind to cells and/or extracellular immunoregulatory factors. The effector element comprises agents such as proteins, nucleic acids, lipids, carbohydrates, glycopeptides, drug moieties (both small molecule drug and biologics), compounds, elements, and isotopes, and fragments thereof.

Although the terms, first, second, third, etc., may be used herein to describe various elements, components, regions, and/or sections, these elements (as well as components, regions, and/or sections) are not to be limited by these terms. Also, the use of such ordinal numbers does not imply a sequence or order unless clearly indicated by the context. Rather, these terms are simply used to distinguish one element from another. Thus, a first element, discussed below, could be termed a second element without departing from the teachings of the exemplary embodiments.

Here, the terms “link,” “couple,” and “conjugates” are used interchangeably to refer to any means of connecting two components either via direct linkage or via indirect linkage between two components.

The term “two consecutive K residues” as used herein refers to two K (or lysine) residues in a peptide core of the present disclosure that are either contiguous to each other (i.e., no other amino acid residues are present between the two K residues); or having other amino acid residues other than lysine inserted between the two K residues. In certain examples, the two consecutive K residues in the present peptide core have one or more amino acid residues other than lysine inserted therebetween, such as the peptide core of SEQ ID NOs: 37 to 40.

The term “polypeptide” as used herein refers to a polymer having at least two amino acid residues. Typically, the polypeptide comprises amino acid residues ranging in length from 2 to about 200 residues; preferably, 2 to 50 residues. Where an amino acid sequence is provided herein, L-, D-, or beta amino acid versions of the sequence are also contemplated. Polypeptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages,” e.g., where the peptide bond is replaced by an α-ester, a β-ester, a thioamide, phosphoramide, carbomate, hydroxylate, and the like.

In certain embodiments, conservative substitutions of the amino acids comprising any of the sequences described herein are contemplated. In various embodiments, one, two, three, four, or five different residues are substituted. The term “conservative substitution” is used to reflect amino acid substitutions that do not substantially alter the activity (e.g., biological or functional activity and/or specificity) of the molecule. Typically, conservative amino acid substitutions involve substitution one amino acid for another amino acid with similar chemical properties (e.g., charge or hydrophobicity). Certain conservative substitutions include “analog substitutions” where a standard amino acid is replaced by a non-standard (e.g., rare, synthetic, etc.) amino acid differing minimally from the parental residue. Amino acid analogs are considered to be derived synthetically from the standard amino acids without sufficient change to the structure of the parent, are isomers, or are metabolite precursors.

In certain embodiments, polypeptides comprising at least 80%, preferably at least 85% or 90%, and more preferably at least 95% or 98% sequence identity with any of the sequences described herein are also contemplated.

“Percentage (%) amino acid sequence identity” with respect to the polypeptide sequences identified herein is defined as the percentage of polypeptide residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two polypeptide sequences was carried out by computer program Blastp (protein-protein BLAST) provided online by Nation Center for Biotechnology Information (NCBI). The percentage amino acid sequence identity of a given polypeptide sequence A to a given polypeptide sequence B (which can alternatively be phrased as a given polypeptide sequence A that has a certain % amino acid sequence identity to a given polypeptide sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in A or B, whichever is shorter.

The term “PEGylated amino acid” as used herein refers to a polyethylene glycol (PEG) chain with one amino group and one carboxyl group. Generally, the PEGylated amino acid has the formula of NH₂—(CH₂CH₂O)ₙ—COOH. In the present disclosure, the value of n ranges from 1 to 20; preferably, ranging from 2 to 12.

As used herein, the term “terminus” with respect to a polypeptide refers to an amino acid residue at the N- or C-end of the polypeptide. With regard to a polymer, the term “terminus” refers to a constitutional unit of the polymer (e.g., the polyethylene glycol of the present disclosure) that is positioned at the end of the polymeric backbone. In the present specification and claims, the term “free terminus” is used to mean the terminal amino acid residue or constitutional unit is not chemically bound to any other molecular.

The term “antigen” or “Ag” as used herein is defined as a molecule that elicits an immune response. This immune response may involve a secretory, humoral and/or cellular antigen-specific response. In the present disclosure, the term “antigen” can be any of a protein, a polypeptide (including mutants or biologically active fragments thereof), a polysaccharide, a glycoprotein, a glycolipid, a nucleic acid, or a combination thereof.

In the present specification and claims, the term “antibody” is used in the broadest sense and covers fully assembled antibodies, antibody fragments that bind with antigens, such as antigen-binding fragment (Fab/Fab′), F(ab′)₂ fragment (having two antigen-binding Fab portions linked together by disulfide bonds), variable fragment (Fv), single chain variable fragment (scFv), bi-specific single-chain variable fragment (bi-scFv), nanobodies, unibodies and diabodies. “Antibody fragments” comprise a portion of an intact antibody, preferably the antigen-binding region or variable region of the intact antibody. Typically, an “antibody” refers to a protein consisting of one or more polypeptides substantially encoded by immunoglobulin genes or fragments of immunoglobulin genes. The well-known immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. A typical immunoglobulin (antibody) structural unit is known to comprise a tetramer. Each tetramer is composed of two identical pairs of polypeptide chains, with each pair having one “light” chain (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively. According to embodiments of the present disclosure, the antibody fragment can be produced by modifying the nature antibody or by de novo synthesis using recombinant DNA methodologies. In certain embodiments of the present disclosure, the antibody and/or antibody fragment can be bispecific, and can be in various configurations. For example, bispecific antibodies may comprise two different antigen binding sites (variable regions). In various embodiments, bispecific antibodies can be produced by hybridoma technique or recombinant DNA technique. In certain embodiments, bispecific antibodies have binding specificities for at least two different epitopes.

The term “specifically binds” as used herein, refers to the ability of an antibody or an antigen-binding fragment thereof, to bind to an antigen with a dissociation constant (Kd) of no more than about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, and/or to bind to an antigen with an affinity that is at least two-folds greater than its affinity to a nonspecific antigen.

The term “treatment” as used herein includes preventative (e.g., prophylactic), curative or palliative treatment; and “treating” as used herein also includes preventative (e.g., prophylactic), curative or palliative treatment. In particular, the term “treating” as used herein refers to the application or administration of the present molecular construct or a pharmaceutical composition comprising the same to a subject, who has a medical condition a symptom associated with the medical condition, a disease or disorder secondary to the medical condition, or a predisposition toward the medical condition, with the purpose to partially or completely alleviate, ameliorate, relieve, delay onset of, inhibit progression of, reduce severity of, and/or reduce incidence of one or more symptoms or features of said particular disease, disorder, and/or condition. Treatment may be administered to a subject who does not exhibit signs of a disease, disorder, and/or condition, and/or to a subject who exhibits only early signs of a disease, disorder and/or condition, for the purpose of decreasing the risk of developing pathology associated with the disease, disorder and/or condition.

The term “effective amount” as used herein refers to the quantity of the present molecular construct that is sufficient to yield a desired therapeutic response. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as particular condition being treated, the physical condition of the patient (e.g., the patient's body mass, age, or gender), the type of subject being treated, the duration of the treatment, the nature of concurrent therapy (if any), and the specific formulations employed and the structure of the compounds or its derivatives. Effective amount may be expressed, for example, as the total mass of active component (e.g., in grams, milligrams or micrograms) or a ratio of mass of active component to body mass, e.g., as milligrams per kilogram (mg/kg).

The terms “application” and “administration” are used interchangeably herein to mean the application of a molecular construct or a pharmaceutical composition of the present invention to a subject in need of a treatment thereof.

The terms “subject” and “patient” are used interchangeably herein and are intended to mean an animal including the human species that is treatable by the molecular construct, pharmaceutical composition, and/or method of the present invention. The term “subject” or “patient” intended to refer to both the male and female gender unless one gender is specifically indicated. Accordingly, the term “subject” or “patient” comprises any mammal, which may benefit from the treatment method of the present disclosure. Examples of a “subject” or “patient” include, but are not limited to, a human, rat, mouse, guinea pig, monkey, pig, goat, cow, horse, dog, cat, bird and fowl. In an exemplary embodiment, the patient is a human. The term “mammal” refers to all members of the class Mammalia, including humans, primates, domestic and farm animals, such as rabbit, pig, sheep, and cattle; as well as zoo, sports or pet animals; and rodents, such as mouse and rat. The term “non-human mammal” refers to all members of the class Mammalis except human.

The present disclosure is based, at least on the construction of the T-E pharmaceuticals that can be delivered to target cells, target tissues or organs at increased proportions relative to the blood circulation, lymphoid system, and other cells, tissues or organs. When this is achieved, the therapeutic effect of the pharmaceuticals is increased, while the scope and severity of the side effects and toxicity is decreased. It is also possible that a therapeutic effector is administered at a lower dosage in the form of a T-E molecule, than in a form without a targeting component. Therefore, the therapeutic effector can be administered at lower dosages without losing potency, while lowering side effects and toxicity.

PART I Multi-Arm Linkers for Treating Specific Diseases

I-(i) Peptide Core for Use in Multi-Arm Linker

The first aspect of the present disclosure pertains to a linker unit that comprises, (1) a center core that comprises 2-15 lysine (K) residues, and (2) 2-15 linking arms respectively linked to the K residues of the center core. The present center core is characterized in having or being linked with an azide group, an alkyne group, a tetrazine group or a strained alkyne group at its N- or C-terminus or between one K residue and its next K residue.

In the preparation of the present linker unit, a PEG chain having a NHS group at one terminus and a functional group (e.g., an NHS, a maleimide, an azide, an alkyne, a tetrazine, or a strained alkyne group) at the other terminus is linked to the K residue of the center core by forming an amide bond between the NHS group of the PEG chain and the amine group of the K residue. In the present disclosure, the PEG chain linked to the K residue is referred to as a linking arm, which has a functional group at the free-terminus thereof.

According to the embodiments of the present disclosure, the center core is a polypeptide that has 8-120 amino acid residues in length and comprises 2 to 15 lysine (K) residues and 1 to 3 conjugating amino acid residues, in which each K residue and the next K residue and a conjugating amino acid residue and its adjacent K residue(s) are separated by a filler sequence and/or a conjugating sequence.

According to some embodiments of the present disclosure, the center core comprises at least one conjugating sequence, which may be disposed at the N- or C-terminus of the center core or between one K residue and its next K residue. According to the embodiment, the conjugating sequence comprises an amino acid residue having a thiol group, an azide group, or an alkyne group (hereinafter, the conjugating amino acid residue). As would be appreciated, when the center core comprises more than one conjugating sequence, these conjugating sequences may comprise the same or different conjugating amino acid residues. For example, in the center core comprising three conjugating sequences, two of the conjugating sequences may respectively comprise the cysteine (C) resides, while the third conjugating sequence may comprise the amino acid residue having the azide or alkyne group. According to the present invention, the conjugating amino acid residue does not directly follow or precede any K residues; that is, there are at least one amino acid residue disposed between the conjugating amino acid residue and the K residues. Preferably, the conjugating amino acid residue is disposed near the center of the conjugating sequence.

According to the preferred embodiments of the present disclosure, the conjugating amino acid residue is not disposed at the N- or C-terminus of the center core. In some embodiments, the conjugating sequence comprising the conjugating amino acid residue is disposed between one K residue and its next K residue. For example, the center core may have the amino acid sequence of KGGSCSGGK (SEQ ID NO: 39), in which the conjugating sequence comprising the C residue is disposed between the K residues. Alternatively, in the case when the conjugating sequence is disposed at the N-terminus of the center core, then the first amino acid residue thereof is an amino acid residue other than the conjugating amino acid residue. For example, the center core may have the amino acid sequence of GSG^{HP}GGKGGSSK (SEQ ID NO: 40), in which the first amino acid residue of the conjugating sequence disposed at the N-terminus of the center core is a glycine (G) residue. Similarly, when the conjugating sequence is disposed at the C-terminus of the center core, then the last amino acid residue thereof is an amino acid residue other than the conjugating amino acid residue.

According to some embodiments of the present disclosure, in addition to the conjugating amino acid residue, the rest of the amino acid residues comprised in the conjugating sequence is selected from the group consisting of, glycine (G), serine (S), arginine (R), histidine (H), aspartic acid (D), glutamic acid (E), threonine (T), asparagine (N), glutamine (Q), proline (P), alanine (A), valine (V), isoleucine (I), leucine (L), methionine (M), phenylalanine (F), tyrosine (Y), and tryptophan (W) residues. According to other embodiments of the present disclosure, the rest of the amino acid residues comprised in the conjugating sequence is selected from the group consisting of, G, S, R, H, D, and E residues. In an alternative example, the rest of the amino acid residues comprised in the conjugating sequence is selected from the group consisting of, R, H, D, and E residues.

Optionally, the present center core comprises one or more filler sequence, each of which is disposed between one K residue and its next K residue or between a conjugating amino acid residue and its adjacent K residue(s). According to the embodiments of the present disclosure, each of the filler sequences independently comprises two or more amino acid residues other than the conjugating amino acid residue.

More specifically, the present disclosure provides three types of filler sequences. The first type of filler sequence is devoid of glycine (G), serine (S), or a combination thereof. Preferably, the amino acid residue of this type of filler sequence is selected from the group consisting of, arginine (R), histidine (H), aspartic acid (D), and glutamic acid (E) residues. For example, the filler sequences may be selected from the group consisting of,

(SEQ ID NO: 1)
SRRS,
(SEQ ID NO: 2)
SRHS,
(SEQ ID NO: 3)
SHHS,
(SEQ ID NO: 4)
SEES,
(SEQ ID NO: 5)
SRSRS,
(SEQ ID NO: 6)
SHSHS,
(SEQ ID NO: 7)
SRSHS,
(SEQ ID NO: 8)
SDSDS,
(SEQ ID NO: 9)
SESES,
(SEQ ID NO: 10)
SRRRS,
(SEQ ID NO: 11)
SHHHS,
(SEQ ID NO: 12)
SRHRS,
(SEQ ID NO: 13)
SDDDS,
(SEQ ID NO: 14)
SEEES,
(SEQ ID NO: 15)
SRDRS,
(SEQ ID NO: 16)
SRSSRS,
(SEQ ID NO: 17)
SRHHRS,
(SEQ ID NO: 18)
SHRRHS,
(SEQ ID NO: 19)
SSDDSS,
and
(SEQ ID NO: 20)
SRDDRS.

The second type of filler sequence comprises glycine (G) and serine (S) residues; preferably, the filler sequence consists of 2-15 residues selected from G, S, and a combination thereof. For example, the filler sequence can be,

GS,
CGS,
GSG,
(SEQ ID NO: 21)
GGGS,
(SEQ ID NO: 22)
GSGS,
(SEQ ID NO: 23)
GGSG,
(SEQ ID NO: 24)
GSGGS,
(SEQ ID NO: 25)
SGGSG,
(SEQ ID NO: 26)
GGGGS,
(SEQ ID NO: 27)
GGSGGS,
(SEQ ID NO: 28)
GGSGGSG,
(SEQ ID NO: 29)
SGSGGSGS,
(SEQ ID NO: 30)
GSGGSGSGS,
(SEQ ID NO: 31)
SGGSGGSGSG,
(SEQ ID NO: 32)
GGSGGSGGSGS,
(SEQ ID NO: 33)
SGGSGGSGSGGS,
(SEQ ID NO: 34)
GGGGSGGSGGGGS,
(SEQ ID NO: 35)
GGGSGSGSGSGGGS,
or
(SEQ ID NO: 36)
SGSGGGGGSGGSGSG.

The filler sequence placed between two lysine residues may be variations of glycine and serine residues in somewhat random sequences and/or lengths. Longer fillers may be used for a polypeptide with fewer lysine residues, and shorter fillers for a polypeptide with more lysine residues. Hydrophilic amino acid residues, such as aspartic acid, glutamate, asparagine, glutamine, arginine, and histidine, may be inserted into the filler sequences together with glycine and serine. As alternatives for filler sequences made up with glycine and serine residues, filler sequences may also be adopted from flexible, soluble loops in common human serum proteins, such as albumin and immunoglobulins.

The third type of filler sequence is a PEGylated amino acid having 2 to 12 repeats of ethylene glycol (EG) unit.

With the similar concept of conjugating sequence, when the center core comprises more than one filler sequence, these filler sequences may belong to the same or different types of filler sequences, and/or comprise the same or different amino acid residues/EG units. For example, each of the filler sequences comprised in the center core may be independently selected from the sequences of SEQ ID NOs: 1-20. Alternatively, one of the filler sequences in the center core is selected from the sequences of SEQ ID NOs: 1-20, and the rest of filler sequences in the center core are independently selected from the sequences of SEQ ID NOs: 21-36. Preferably, at least one of the filler sequence is selected from the sequences of SEQ ID NOs: 1-20.

When the center core comprises both the filler sequence and the conjugating sequence, it is preferred that the length of the conjugating sequence is at least twice the length of the filler sequence. In essence, when a conjugating sequence is placed between two K residues, the conjugating amino acid residue in the conjugating sequence is separated from its adjacent K residues by peptides that are as long as or longer than a filler sequence.

As would be appreciated, the present center core may not comprise the conjugating sequence. In this case, each of the K residue and its next K reside in the center core is separated by the filler sequences independently selected from type I, type II and/or type III filler sequences mentioned above.

The amino acid residue having an azide group can be, L-azidohomoalanine (AHA), 4-azido-L-phenylalanine, 4-azido-D-phenylalanine, 3-azido-L-alanine, 3-azido-D-alanine, 4-azido-L-homoalanine, 4-azido-D-homoalanine, 5-azido-L-ornithine, 5-azido-d-ornithine, 6-azido-L-lysine, or 6-azido-D-lysine.

Exemplary amino acid having an alkyne group includes, but is not limited to, L-HPG, D-HPG, or β-HPG.

It is noted that many of the amino acids containing an azide or alkyne group in their side chains and PEGylated amino acids are available commercially in t-boc (tert-butyloxycarbonyl)- or Fmoc (9-fluorenylmethyloxycarbonyl)-protected forms, which are readily applicable in solid-phase peptide synthesis.

Alternatively, the present center core is linked with a coupling arm, which has a functional group (e.g., an azide group, an alkyne group, a tetrazine group, or a strained alkyne group) at the free-terminus thereof (that is, the terminus that is not linked to the center core). In these cases, the conjugating amino acid residue of the present center core comprises a cysteine residue. To prepare a linker unit linked with a coupling arm, a PEG chain having a maleimide group at one terminus and a functional group at the other terminus is linked to the cysteine residue of the center core via thiol-maleimide reaction occurred between the maleimide group of the PEG chain and the thiol group of the cysteine residue. In the present disclosure, the PEG chain linked to the cysteine residue of the center core is referred to as the coupling arm, which has a functional group at the free-terminus thereof.

Preferably, the coupling arm has a tetrazine group or a strained alkyne group (e.g., a cyclooctene or cyclooctyne group) at the free-terminus thereof. These coupling arms have 2-12 EG units. According to the embodiments of the present disclosure, the tetrazine group is 1,2,3,4-tetrazine, 1,2,3,5-tetrazine, 1,2,4,5-tetrazine, or derivatives thereof. The strained alkyne group may be a cyclooctene or a cyclooctyne group. According to the working examples of the present disclosure, the cyclooctene group is a TCO group; example of cyclooctyne group includes, but is not limited to, DBCO, DIFO, BCN, and DICO group. According to some embodiments of the present disclosure, the tetrazine group is 6-methyl-tetrazine. The polypeptide may also be synthesized using recombinant technology by expressing designed gene segments in bacterial or mammalian host cells. It is preferable to prepare the polypeptide as recombinant proteins if the core has high numbers of lysine residues with considerable lengths. As the length of a polypeptide increases, the number of errors increases, while the purity and/or the yield of the product decrease, if solid-phase synthesis was adopted. To produce a polypeptide in bacterial or mammalian host cells, a filler sequence or a conjugating sequence may be placed between two K residues. Since AHA and HPG are not natural amino acids encoded by the genetic codes, 1 to 2 cysteine residues is placed at the N-terminal, C-terminal or another positions in the recombinant polypeptide. After the recombinant proteins are expressed and purified, the cysteine residues are then reacted with short bifunctional cross-linkers, which have maleimide group at one end, which reacts with SH group of cysteine residue, and alkyne, azide, tetrazine, or strained alkyne at the other end.

The synthesis of a polypeptide using PEGylated amino acids involves fewer steps than that with regular amino acids such as glycine and serine resides. In addition, PEGylated amino acids with varying lengths (i.e., numbers of repeated ethylene glycol units) may be employed, offering flexibility for solubility and spacing between adjacent amino groups of lysine residues. In addition to PEGylated amino acids, the center cores may also be constructed to comprise artificial amino acids, such as D-form amino acids, homo-amino acids, N-methyl amino acids, etc. Preferably, the PEGylated amino acids with varying lengths of polyethylene glycol (PEG) are used to construct the center core, because the PEG moieties contained in the amino acid molecules provide conformational flexibility and adequate spacing between conjugating groups, enhance aqueous solubility, and are generally weakly immunogenic. The synthesis of PEGylated amino acid-containing center core is similar to the procedures for the synthesis of regular polypeptides.

Optionally, for stability purpose, the present center core has an acetyl group to block the amino group at its N-terminus.

As could be appreciated, the number of the linking arms linked to the center core is mainly determined by the number of lysine resides comprised in the center core. Since there are at least two lysine residues comprised in the present center core, the present linker unit may comprise a plurality of linking arms.

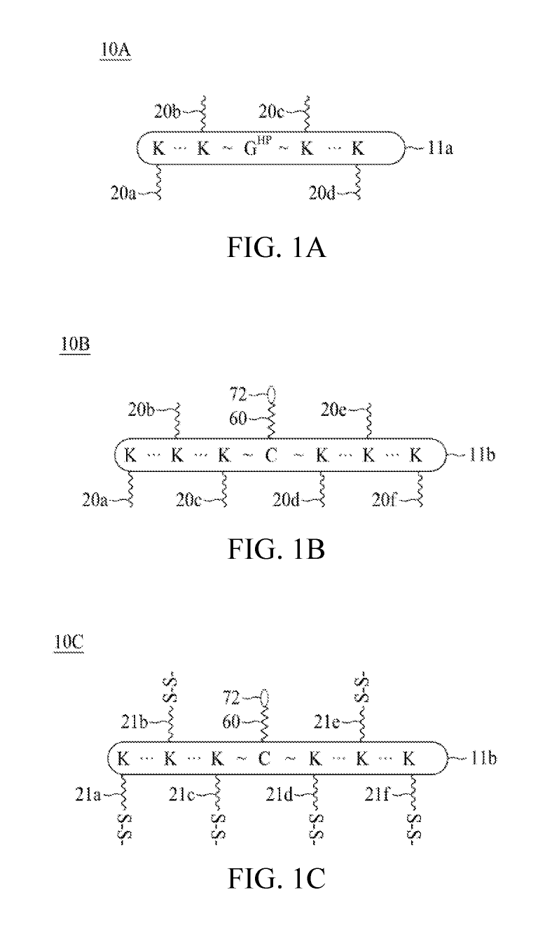

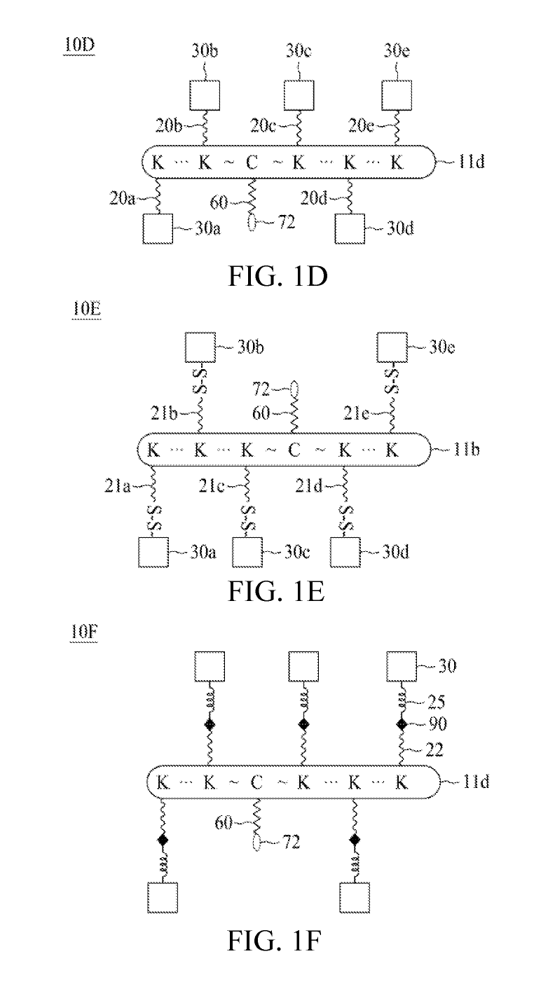

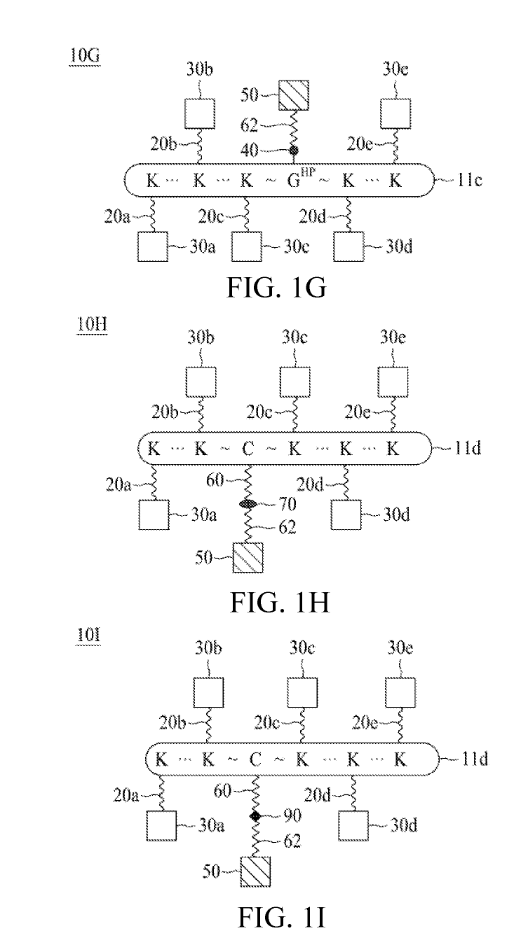

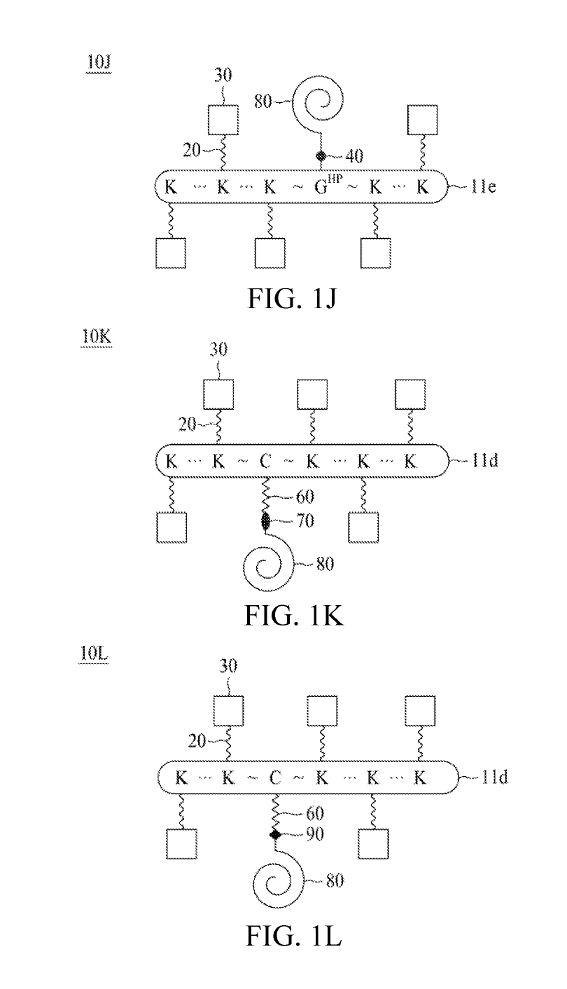

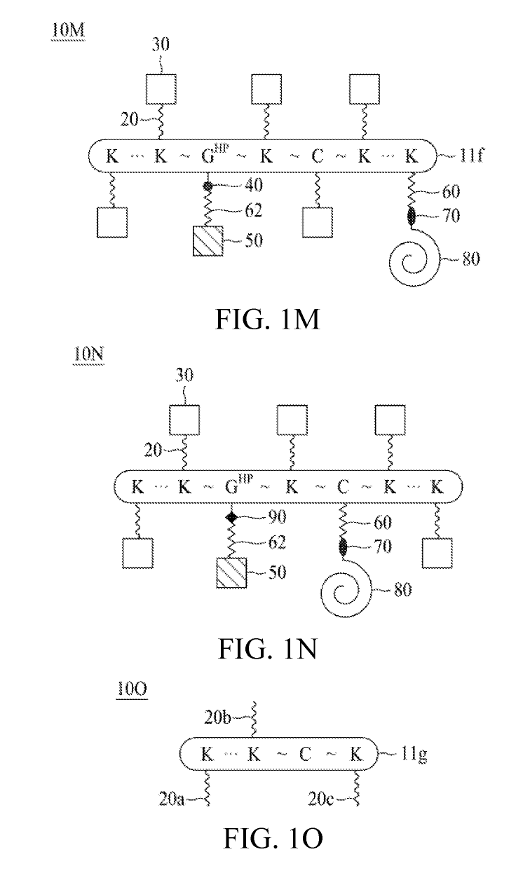

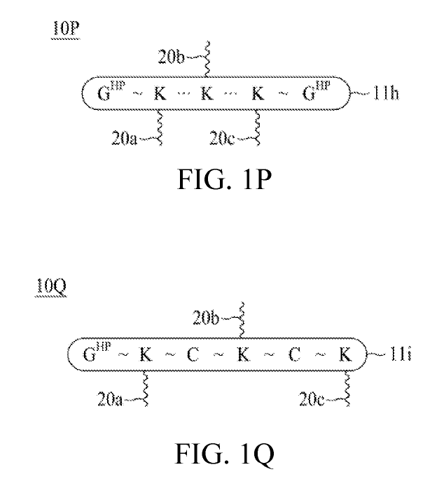

Reference is now made to FIG. 1A. As illustrated, the linker unit 10A comprises a center core 11a comprising four lysine (K) residues, in which the first and second K residues (1^{st}-2^{nd} K residues) and the third and fourth K residues (3^{rd}-4^{th} K residues) are respectively separated by filler sequences (denoted by the dots throughout the drawings), while the second and third K residues (2^{nd}-3^{rd} K residues) are separated by the conjugating sequence (denoted by the symbol˜throughout the drawings) comprising one HPG (G^{HP}) residue. The filler sequences and the conjugating sequence between any two K residues may comprise the same or different amino acid sequences. In this example, four linking arms 20a-20d are linked to the lysine residues by forming an amide linkage between the NHS group and the amine group of the lysine residue, respectively.

FIGS. 1O-1Q provide alternative examples of the center core. In FIG. 1O, the center core 11g of linker unit 10O comprises three K residues; accordingly, three linking arms 20a-20c can be respectively linked to the K residues. In this example, the 1^{st}-2^{nd} K residues are separated by the filler sequence, and the 2^{nd}-3^{rd} K residues are separated by the conjugating sequence comprising one C residue. FIG. 1P provides a linker unit 10P, in which the center core 11h comprises three K residues respectively separated by filler sequences. In addition to the filler sequences, the center core 11h further comprises two conjugating sequences respectively disposed at the N terminus and the C terminus thereof, in which each of the conjugating sequences comprises one HPG (G^{HP}) residue. FIG. 1Q provides a linker unit 10Q, in which the center core 11i comprises three K residues. Each K residue and its next K residue are separated by the conjugating sequence comprising one C residue. Further, one conjugating sequence comprising one HPG (G^{HP}) residue is disposed at the N-terminus of the center core 11i.

As could be appreciated, certain features discussed above regarding the linker units 10A, 10O, 10P and 10Q, or any other following linker units are common to other linker units disclosed herein, and hence some or all of these features are also applicable in the following examples, unless it is contradictory to the context of a specific embodiment. However, for the sake of brevity, these common features may not be explicitly repeated below.

FIG. 1B provides a linker unit 10B according to another embodiment of the present disclosure. The center core 11b comprises six lysine (K) residues, in which 1^{st}-2^{nd}, 2^{nd}-3^{rd}, 4^{th}-5^{th}, and 5^{th}-6^{th} K residues are respectively separated by the filler sequences, and the 3^{rd}-4^{th} K residues are separated by the conjugating sequence comprising one C residue. In this example, the linker unit 10B comprises six linking arms 20a-20f that are respectively linked to the lysine residues. According to the embodiments of the present disclosure, the linking arm is a PEG chain having 2-20 repeats of EG units.

Unlike the linker unit 10A of FIG. 1A, the linker unit 1B further comprises a coupling arm 60. As discussed above, a PEG chain having a maleimide group at one end and a functional group at the other end is used to form the coupling arm 60. In this way, the coupling arm 60 is linked to the cysteine residue of the center core 11b via thiol-maleimide reaction. In this example, the functional group at the free terminus of the coupling arm 60 is a tetrazine group 72. According to the embodiments of the present disclosure, the coupling arm is a PEG chain having 2-12 repeats of EG units.

When the release of effector elements at the targeted site is required, a cleavable bond can be installed in the linking arm. Such a bond is cleaved by acid/alkaline hydrolysis, reduction/oxidation, or enzymes. One embodiment of a class of cleavable PEG chains that can be used to form the coupling arm is NHS-PEG₂₋₂₀-S—S-maleimide, where S—S is a disulfide bond that can be slowly reduced, while the NHS group is used for conjugating with the amine group of the center core, thereby linking the PEG chain onto the center core. The maleimide group at the free terminus of the linking arm may be substituted by an azide, alkyne, tetrazine, or strained alkyne group. According to some embodiments of the present disclosure, the linking arm is a PEG chain, which has 2-20 repeats of EG units with a disulfide linkage at the free terminus thereof (i.e., the terminus that is not linked with the center core). Reference is now made to FIG. 1C, in which each of the five linking arms 21a-21f respectively linked to the K resides of the center core 11b is a PEG chain with a disulfide linkage at the free terminus of the linking arm.

According to the embodiments of the present disclosure, the linking arm linked to the K residue of the center core has a functional group (i.e., a maleimide, an NHS, an azide, an alkyne, a tetrazine, or a strained alkyne group) at its free terminus. Preferably, when the free terminus of the linking arm is an azide, alkyne, or cyclooctyne group, then the conjugating sequence of the center core comprises a cysteine residue, and the free terminus of the coupling arm is a tetrazine or cyclooctene group. Alternatively, when the free terminus of the linking arm is a tetrazine group or cyclooctene group, then (1) the conjugating sequence of the center core comprises an azide or alkyne group, or (2) the conjugating sequence of the center core comprises a cysteine residue, and the free terminus of the coupling arm is an azide, the alkyne, or the cyclooctyne group.

Depending on the functional group (i.e., a maleimide, an NHS, an azide, an alkyne, a tetrazine, or a strained alkyne group) present at the free terminus of the linking arm, it is feasible to design a functional element (such as, a targeting element, an effector element, or an element for improving the pharmacokinetic property) with a corresponding functional group, so that the functional element may linked to the free terminus of the linking arm via any of the following chemical reactions,

(1) forming an amide bond therebetween: in this case, the linking arm has an NHS group at the free terminus, and the functional element has an amine group;

(2) the thiol-maleimide reaction: in this case, the linking arm has a maleimide group at the free terminus, and the functional element has an thiol group;

(3) the Copper(I)-catalyzed alkyne-azide cycloaddition reaction (CuAAC reaction, or the “click” reaction for short): one of the free terminus of the linking arm and the functional element has an azide group, while the other has an alkyne group; the CuAAC reaction is exemplified in Scheme 1;

(4) the inverse electron demand Diels-Alder (iEDDA) reaction: one of the free terminus of the linking arm and the functional element has a tetrazine group, while the other has a cyclooctene group; the iEDDA reaction is exemplified in Scheme 2; or

(5) the strained-promoted azide-alkyne click chemistry (SPAAC) reaction: one of the free terminus of the linking arm and the functional element has an azide group, while the other has an cyclooctyne group; the SPAAC reaction is exemplified in Scheme 3.

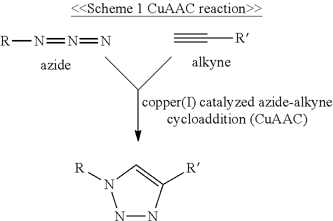

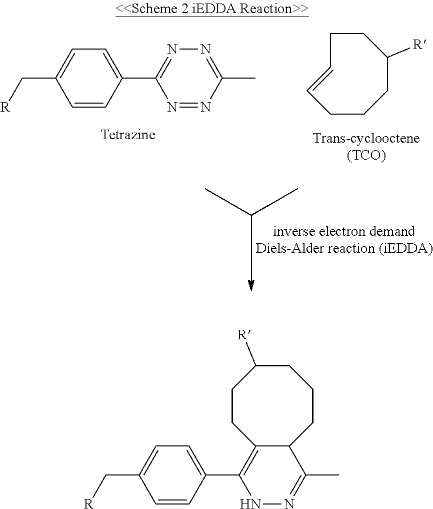

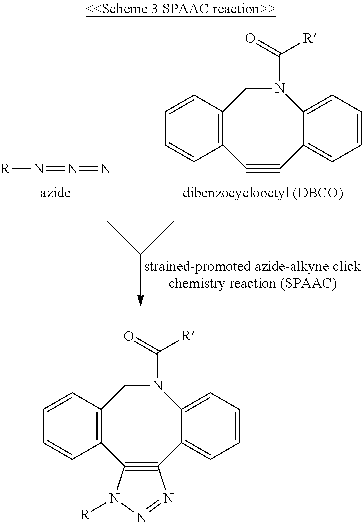

The CuAAC reaction yields 1,5 di-substituted 1,2,3-triazole. The reaction between alkyne and azide is very selective and there are no alkyne and azide groups in natural biomolecules. Furthermore, the reaction is quick and pH-insensitive. It has been suggested that instead of using copper (I), such as cuprous bromide or cuprous iodide, for catalyzing the click reaction, it is better to use a mixture of copper (II) and a reducing agent, such as sodium ascorbate to produce copper (I) in situ in the reaction mixture. Alternatively, the second element can be linked to the N- or C-terminus of the present center core via a copper-free reaction, in which pentamethylcyclopentadienyl ruthenium chloride complex is used as the catalyst to catalyze the azide-alkyne cycloaddition.

For the sake of illustration, the functional elements linked to the linking arms are referred to as the first elements. As could be appreciated, the number of the first elements carried by the present linker unit depends on the number of K residues of the center core (and thus, the number of the linking arms). Accordingly, one of ordinary skill in the art may adjust the number of the first elements of the linker unit as necessary, for example, to achieve the desired targeting or therapeutic effect.

An example of a linker unit 10D having the first elements is illustrated FIG. 1D. Other than the features discussed hereafter, FIG. 1D is quite similar to FIG. 1B. First, there are five K residues in the center core lid, and accordingly, five linking arms 20a-20e are linked thereto, respectively. Second, the linker unit 10D has five first elements 30a-30e linked to each of the linking arms 20a-20e. As discussed below, the optional tetrazine group 72 allows for the conjugation with an additional functional element, another molecular construct (see, Part II or Part III below).

FIG. 1E provides an alternative example, in which the linker unit 10E has a similar structure with the linker unit 10D, except that each of the linking arms 21a-21e has a disulfide linkage at the element-linking terminus thereof (i.e., the terminus that is linked with each of the first elements 30a-30e).

Alternatively, the present linker unit further comprises a plurality of connecting arms, each of which has a functional group (i.e., a maleimide, an NHS, an azide, an alkyne, a tetrazine, or a strained alkyne group) at one terminus, and an NHS or a maleimide group at the other terminus. Using a reaction that is similar to those occurred between the first element and the linking arm, the connecting arm may be linked to the linking arm with the corresponding functional group either via forming an amide bond therebetween, or via the thiol-maleimide, CuAAC, iEDDA or SPAAC reaction. The connecting arm linked to the linking arm thus has the NHS or the maleimide group at its free terminus (or the element-linking terminus; i.e., the terminus that is not linked with the linking arm); then, the first element is linked to the element-linking terminus of the connecting arm via forming an amide bond therebetween or via the thiol-maleimide reaction.

Reference is now made to FIG. 1F, in which the linking arm is linked to the K residue of the center core 11d as described in FIG. 1D. Compared with the linker unit 10D, the linker unit 10F further comprises a connecting arm 25, which is linked to the linking arms 22 via the SPAAC reaction. Then, the first element 30 is linked to the connecting arm 25 either via forming the amide bond therebetween or via the thiol-maleimide reaction. The diamond 90 as depicted in FIG. 1F represents the chemical bond resulted from the SPAAC reaction occurred between the linking arm 22 and the connecting arm 25.

According to some embodiments of the present disclosure, the connecting arm is a PEG chain having 2-20 repeats of EG units. Alternatively, the connecting arm is a PEG chain having 2-20 repeats of EG units with a disulfide linkage at the element-linking terminus thereof (i.e., the free terminus that is not linked with the linking arm).

In one working example, the connecting arm has three repeats of EG units, as well as a disulfide linkage at the free terminus (also referred to as the element-linking terminus) of the connecting arm. In this case, the first element linked to the element-linking terminus of the connecting arm can be efficiently released from the present linker unit by the treatment of a reductant.

In order to increase the intended or desired effect (e.g., the therapeutic effect), the present linker unit may further comprise a second element in addition to the first element. For example, the second element can be either a targeting element or an effector element. In optional embodiments of the present disclosure, the first element is an effector element, while the second element may be another effector element, which works additively or synergistically with or independently of the first element. Still optionally, the first and second elements exhibit different properties; for example, the first element is a targeting element, and the second element is an effector element, and vice versa. Alternatively, the first element is an effector element, and the second element is an element capable of improving the pharmacokinetic property of the linker unit, such as solubility, clearance, half-life, and bioavailability. The choice of a particular first element and/or second element depends on the intended application in which the present linker unit (or multi-arm linker) is to be used. Examples of these functional elements are discussed below in Part I-(ii) of this specification.

Structurally, the second element is linked to the azide, alkyne, tetrazine, or strained alkyne group within the conjugating sequence of the center core. Specifically, the second element may be optionally conjugated with a short PEG chain (preferably having 2-12 repeats of EG units) and then linked to the conjugating sequence comprising an azide group or an alkyne group (e.g., AHA residue or HPG residue). Alternatively, the second element may be optionally conjugated with the short PEG chain and then linked to the coupling arm of the center core.

According to some embodiments of the present disclosure, the conjugating sequence of the center core comprises an amino acid having an azide group (e.g., the AHA residue); and accordingly, a second element having an alkyne group is linked to the conjugating sequence of the center core via the CuAAC reaction. According to other embodiments of the present disclosure, the conjugating sequence of the center core comprises an amino acid having an alkyne group (e.g., the HPG residue); and a second element having an azide group is thus capable of being linked to the conjugating sequence of the center core via the CuAAC reaction.

FIG. 1G provides an example of the present linker unit 10G carrying a plurality of first elements and one second element. In this example, the center core 11c comprises five lysine (K) residues, in which 1^{st}-2^{nd}, 2^{nd}-3^{rd}, 4^{th}-5^{th} K residues are separated by the filler sequences, and the 3^{rd}-4^{th} K residues are separated by the conjugating sequence comprising one HPG (G^{HP}) residue. Five linking arms 20a-20e are respectively linked to the five K residues of the center core 11c; and five first elements 30a-30e are respectively linked to said five linking arms 20a-20e via the thiol-maleimide reaction. In addition to the first elements, the linker unit 10G further comprises one second element 50 that is linked to one end of a short PEG chain 62. Before being conjugated with the center core 11c, the other end of the short PEG chain 62 has an azide group. In this way, the azide group may reacted with the HPG residue that having an alkyne group via CuAAC reaction, so that the second element 50 is linked to the center core 11c. The solid dot 40 depicted in FIG. 1G represents the chemical bond resulted from the CuAAC reaction occurred between the HPG residue and the azide group.

Alternatively, the second element is linked to the center core via a coupling arm. According to certain embodiments of the present disclosure, the coupling arm has a tetrazine group, which can be efficiently linked to a second element having a TCO group via the iEDDA reaction. According to other embodiments of the present disclosure, the coupling arm has a TCO group, which is capable of being linked to a second element having a tetrazine group via the iEDDA reaction. In the iEDDA reaction, the strained cyclooctenes that possess a remarkably decreased activation energy in contrast to terminal alkynes is employed, and thus eliminate the need of an exogenous catalyst.

Reference is now made to FIG. 1H, in which the center core 11d of the linker unit 10H comprises five lysine (K) residues, in which 1^{st}-2^{nd}, 3^{rd}-4^{th}, 4^{th}-5^{th} K residues are separated by the filler sequences, and the 2^{nd}-3^{rd} K residues are separated by the conjugating sequence comprising one C residue. As depicted in FIG. 1H, five linking arms 20a-20e are respectively linked to the five K residue of the center core lid, and then five first elements 30a-30e are respectively linked to the five linking arms 20a-20e via thiol-maleimide reactions. The C residue is linked to the coupling arm 60, which, before being conjugated with the second element, comprises a tetrazine group or a TCO group at its free-terminus. In this example, a second element 50 linked with a short PEG chain 62 having a corresponding TCO or tetrazine group can be linked to the coupling arm 60 via the iEDDA reaction. The ellipse 70 as depicted in FIG. 1H represents the chemical bond resulted from the iEDDA reaction occurred between the coupling arm 60 and the short PEG chain 62.

According to other embodiments of the present disclosure, before the conjugation with a second element, the coupling arm has an azide group. As such, the coupling arm can be linked to the second element having a cyclooctyne group (e.g., the DBCO, DIFO, BCN, or DICO group) at the free-terminus of a short PEG chain via SPAAC reaction, and vice versa.

Reference is now made to FIG. 1I, in which the linker unit 10I has a structure similar to the linker unit 10H of FIG. 1H, except that the coupling arm 60 comprises an azide or a cyclooctyne group (e.g., the DBCO, DIFO, BCN, or DICO group), instead of the tetrazine or TCO group. Accordingly, the second element 50 linked with a short PEG chain 62 may have a corresponding cyclooctyne (e.g., DBCO, DIFO, BCN, or DICO) or azide group, so that it can be linked to the coupling arm 60 via the SPAAC reaction. The diamond 90 as depicted in FIG. 1I represents the chemical bond resulted from the SPAAC reaction occurred between the coupling arm 60 and the short PEG chain 62.

FIG. 1J provides an alternative example of the present linker unit (linker unit 10J), in which five first elements 30 are respectively linked to the lysine residues via the linking arms 20, and the HPG (G^{HP}) residue of the center core 11e is linked with a PEG chain 80 via the CuAAC reaction. The solid dot 40 depicted in FIG. 1J represents the chemical bond resulted from the CuAAC reaction occurred between the HPG residue and the PEG chain 80.

FIG. 1K provides another example of the present disclosure, in which the center core 11d comprises a cysteine residue that is linked to a coupling arm 60. A PEG chain 80 can be efficiently linked to the coupling arm 60 via the iEDDA reaction. The ellipse 70 of the linker unit 10K represents the chemical bond resulted from the iEDDA reaction occurred between the coupling arm 60 and the PEG chain 80.

FIG. 1L provides an alternative example of the present linker unit, in which the linker unit 10L has a structure similar to the linker unit 10J of FIG. 1J, except that the PEG chain 80 is linked to the coupling arm 60 via the SPAAC reaction. The diamond 90 depicted in FIG. 1L represents the chemical bond resulted from the SPAAC reaction occurred between the coupling arm 60 and the PEG chain 80.

According to some embodiments of the present disclosure, in addition to the first and second elements, the present linker unit further comprises a third element. In this case, the center core comprises two conjugating sequences, in which one of the conjugating sequences comprises an amino acid having an azide group or an alkyne group, while the other of the conjugating sequences comprises a cysteine residue. The lysine residues of the center core are respectively linked with the linking arms, each of which has a maleimide group at its free terminus; whereas the cysteine residue of the center core is linked with the coupling arm, which has a tetrazine group or a strained alkyne group at its free terminus. As described above, the first element is therefore linked to the linking arm via the thiol-maleimide reaction, and the second element is linked to the coupling arm via the iEDDA reaction. Further, a third element is linked to the amino acid having an azide group or an alkyne group via the CuAAC reaction or SPAAC reaction.

Reference is now made to the linker unit 10M of FIG. 1M, in which the center core 11f comprises an HPG (G^{HP}) residue and a cysteine residue. The linking arms 20 and the coupling arm 60 are respectively linked to the lysine (K) residues and the cysteine (C) residue of the center core 11f. Further, five first elements 30 are respectively linked to the five linking arms 20, the second element (i.e., the PEG chain) 80 is linked to the coupling arm 60, and the third element 50 is linked to the HPG residue via the short PEG chain 62. The solid dot 40 indicated the chemical bond resulted from the CuAAC reaction occurred between the HPG residue and the short PEG chain 62; while the ellipse 70 represents the chemical bond resulted from the iEDDA reaction occurred between the coupling arm 60 and the PEG chain 80.

FIG. 1N provides another embodiment of the present disclosure, in which the linker unit 10N has the similar structure with the linker unit 10M of FIG. 1M, except that the short PEG chain 62 is linked with the HPG residue via the SPAAC reaction, instead of the iEDDA reaction. The diamond 90 in FIG. 1N represents the chemical bond resulted from the SPAAC reaction occurred between the short PEG chain 62 and the HPG residue.

In the preferred embodiments of this disclosure, the linking arms have a maleimide group in the free terminus for conjugating with first elements having the sulfhydryl group via the thiol-maleimide reaction. Also, there is one cysteine residue or an amino acid residue with an azide or alkyne group disposed at the conjugating sequence of the peptide core for attaching a coupling arm for linking a second element.

It is conceivable for those skilled in the arts that variations may be made. A conjugating group, other than maleimide, such as azide, alkyne, tetrazine, or strained alkyne may be used for the free terminus of the linking arms, for linking with first elements with a CuAAC, iEDDA, or SPAAC reaction. Also the cysteine residue (or an amino acid residue with an azide or alkyne group) of the peptide core needs not to be at the N- or C-terminus. Furthermore, two or more of such residues may be incorporated in the peptide core to attach multiple coupling arms for linking a plural of second elements.

I-(ii) Functional Elements Suitable for Use in Multi-Arm Linker

In the case where the linker unit (or multi-arm linker) comprises only the first element but not the second and/or third element(s), the first element is an effector element that may elicit a therapeutic effect in a subject. On the other hand, when the present linker unit comprises elements in addition to first element(s), then at least one of the elements is an effector element, while the other may be another effector element, a targeting element, or an element capable of enhancing one or more pharmacokinetic properties of the linker unit (e.g., solubility, clearance, half-life, and bioavailability). For example, the linker unit may have two different kinds of effector element, one effector element and one targeting element or one pharmacokinetic property-enhancing element, two different kinds of targeting elements and one kind of effector element, two different kinds of effector elements and one kind of targeting element, or one kind of targeting element, one kind of effector element and one element capable of improving the pharmacokinetic property of the linker unit.

For the purpose of treating an immune disorder, the present linker unit comprises two functional element, in which the first element is a single-chain variable fragment (scFv) specific for a cytokine or a receptor of the cytokine; or a soluble receptor of the cytokine; and the second element is an scFv specific for a tissue-associated extracellular matrix protein. According to one embodiment of the present disclosure, the tissue-associated extracellular matrix protein is selected from the group consisting of α-aggrecan, collagen I, collagen II, collagen III, collagen V, collagen VII, collagen IX, and collagen XI; the cytokine is selected from the group consisting of tumor necrosis factor-α (TNF-α), interleukin-17 (IL-17), IL-1, IL-6, IL-12/IL-23, and B cell activating factor (BAFF); the receptor of the cytokine is specific for IL-6 or IL-17; and the soluble receptor of the cytokine is specific for TNF-α or IL-1.

For the treatment of a diffused tumor, the first element of the present linker unit is an scFv specific for a first cell surface antigen, and the second element of the present linker unit is an scFv specific for a second cell surface antigen. According to one embodiment of the present disclosure, the first cell surface antigen is selected from the group consisting of, CD5, CD19, CD20, CD22, CD23, CD27, CD30, CD33, CD34, CD37, CD38, CD43, CD72a, CD78, CD79a, CD79b, CD86, CD134, CD137, CD138, and CD319; and the second cell surface antigen is CD3 or CD16a.

According to some embodiments of the present disclosure, the present linker unit provides a therapeutic benefit in the treatment of a solid tumor. In these embodiments, the first element of the present linker unit is a peptide hormone, a growth factor, or an scFv specific for a tumor-associated antigen; and the second element of the present linker unit is an scFv specific for a cell surface antigen. More specifically, the peptide hormone is secretin, cholecystokinin (CCK), somatostatin, or thyroid-stimulating hormone (TSH); the growth factor is selected from the group consisting of epidermal growth factor (EGF), mutant EGF, epiregulin, heparin-binding epidermal growth factor (HB-EGF), vascular endothelial growth factor A (VEGF-A), basic fibroblast growth factor (bFGF), and hepatocyte growth factor (HGF); the tumor-associated antigen is selected from the group consisting of human epidermal growth factor receptor (HER1), HER2, HER3, HER4, carbohydrate antigen 19-9 (CA 19-9), carbohydrate antigen 125 (CA 125), carcinoembryonic antigen (CEA), mucin 1 (MUC 1), ganglioside GD2, melanoma-associated antigen (MAGE), prostate-specific membrane antigen (PSMA), prostate stem cell antigen (PSCA), mesothelin, mucine-related Tn, Sialyl Tn, Globo H, stage-specific embryonic antigen-4 (SSEA-4), and epithelial cell adhesion molecule (EpCAM); and the cell surface antigen is CD3 or CD16a.

According to certain embodiments of the present disclosure, the present linker unit is useful in treating an osteoporosis disease, in which an scFv specific for receptor activator of nuclear factor κB (RANKL) is employed as the first element; and an scFv specific for collagen I or osteonectin serves as the second element.

According to other embodiments of the present disclosure, the linker unit suitable for the treating an age-related macular degeneration (AMD) comprises two element, in which the first element is an scFv specific for VEGF-A; and the second element is a long PEG chain having a molecular weight of about 20,000 to 50,000 daltons.

Another disorder preventable or treatable by the present invention is central nervous system (CNS) disease and/or infectious disease. According to one embodiment, the first element of the linker unit is fingolimod, fingolimod phosphate, interferon-β, or an scFv specific for integrin-α4, β-amyloid, a viral protein, or a bacterial protein; and the second element of the linker unit is an scFv specific for transferrin receptor, CD32 or CD16b. Examples of viral proteins include, but are not limited to, F protein of respiratory syncytia virus (RSV), gp120 protein of human immunodeficiency virus type 1 (HIV-1), hemagglutinin A (HA) protein of influenza A virus, and glycoprotein of cytomegalovirus. Illustrative examples of bacterial protein include endotoxin of Gram(-) bacteria, surface antigen of Clostridium difficile, lipoteichoic acid of Saphylococcus aureus, anthrax toxin of Bacillus anthracis, and Shiga-like toxin type I or II of Escherichia coli.

In one embodiment of the present disclosure, the present linker unit is configured to preventing the formation of blood clot and/or treating thrombosis. In the embodiment, the first element is an scFv specific for fibrin; and the second element is a tissue plasminogen activator or an inhibitor of Factor Xa or thrombin. According to the embodiment, the tissue plasminogen activator is alteplase, reteplase, tenecteplase, or lanoteplase; the inhibitor of Factor Xa is apixaban, edoxaban, or rivaroxaban; and the inhibitor of thrombin is argatroban or melagatran.

In another embodiment of the present disclosure, the present linker unit is useful in treating a transplantation rejection, in which the first element is an scFv specific for human leukocyte antigen (HLA)-A, HLA-B or HLV-C, and the second element is a cell surface antigen, or an inhibitor of mammalian target of rapamycin (mTOR) or calcineurin. Non-limiting example of the cell surface antigen includes, cytotoxic T lymphocyte associated protein 4 (CTLA-4), and programmed death-ligand 1 (PD-L1). The inhibitor of mTOR can be sirolimus or everolimus; and the inhibitor of calcineurin can be tacrolimus.

I-(iii) Use of Multi-Arm Linker

The present disclosure also pertains to method for treating various diseases using the suitable linker unit, including an immune disorder, a diffused tumor, a solid tumor, an osteoporosis disease, an age-related macular degeneration (AMD), a central nervous system (CNS) disease, an infectious disease, blood clot-related disease (e.g., thrombosis) and transplantation rejection. Generally, the method comprises the step of administering to a subject in need of such treatment an effective amount of the linker unit according to embodiments of the present disclosure.

Compared with previously known therapeutic constructs, the present linker unit discussed in Part I is advantageous in two points:

(1) The number of the functional elements may be adjusted in accordance with the needs and/or applications. The present linker unit may comprise two elements (i.e., the first and second elements) or three elements (i.e., the first, second, and third elements) in accordance with the requirements of the application (e.g., the disease being treated, the route of administration of the present linker unit, and the binding avidity and/or affinity of the antibody carried by the present linker unit). For example, when the present linker unit is directly delivered into the tissue/organ (e.g., the treatment of eye), one element acting as the effector element may be enough, thus would eliminate the need of a second element acting as the targeting element. However, when the present linker unit is delivered peripherally (e.g., oral, enteral, nasal, topical, transmucosal, intramuscular, intravenous, or intraperitoneal injection), it may be necessary for the present linker unit to simultaneously comprise a targeting element that specifically targets the present linker unit to the lesion site; and an effector element that exhibits a therapeutic effect on the lesion site. For the purpose of increasing the targeting or treatment efficacy or increasing the stability of the present linker unit, a third element (e.g., a second targeting element, a second effector element, or a PEG chain) may be further included in the present linker unit.

(2) The first element is provided in the form of a bundle. As described above, the number of the first element may vary with the number of lysine residue comprised in the center core. If the number of lysine residue in the center core ranges from 2 to 15, then at least two first elements may be comprised in each linker unit. Thus, instead of providing one single molecule (e.g., cytotoxic drug and antibody) as traditional therapeutic construct or method may render, the present linker unit is capable of providing more functional elements (either as targeting elements or as effector elements) at one time, thereby greatly improves the therapeutic effect.

In certain therapeutic applications, it is desirable to have a single copy of a targeting or effector element. For example, a single copy of a targeting element can be used to avoid unwanted effects due to overly tight binding. This consideration is relevant, when the scFv has a relatively high affinity for the targeted antigen and when the targeted antigen is a cell surface antigen on normal cells, which are not targeted diseased cells. As an example, in using scFv specific for CD3 or CD16a to recruit T cells or NK cells to kill targeted cells, such as thyroid gland cells in patients with Graves' disease, a single copy of the scFv specific for CD3 or CD16a is desirable, so that unwanted effects due to cross-linking of the CD3 or CD16a may be avoided. Similarly, in using scFv specific for CD32 or CD16b to recruit phagocytic neutrophils and macrophages to clear antibody-bound viral or bacterial particles or their products, a single copy of scFv may be desirable. Also, in using scFv specific for transferrin receptor to carry effector drug molecules to the BBB for treating CNS diseases, a single copy of scFv specific for transferrin receptor is desirable. In still another example, it is desirable to have only one copy of long-chain PEG for enhancing pharmacokinetic properties. Two or more long PEG chains may cause tangling and affect the binding properties of the targeting or effector elements.

PART II Joint-Linker Molecular Constructs for Treating Specific Diseases

Another aspect of the present disclosure pertains to a molecular construct comprising at least two linker units, in which one linker unit carries one or more targeting element, whereas another other linker unit carries one or more effector elements or pharmacokinetic property-enhancing elements. In the present disclosure, molecular constructs with both the targeting and effector moieties (whether a therapeutic or pharmacokinetic one) are referred to as joint-linker molecular constructs. According to various embodiments of the present disclosure, each of the linker unit comprised in such joint-linker molecular constructs is a peptide core-based discussed above in Part I of the present disclosure. According to certain embodiments of the present disclosure, at least one of the linker units of the present molecular construct comprises the polypeptide core. Preferably, at least two linker units of the present molecular construct comprise the polypeptide cores. More preferably, all the linker units of present molecular construct respectively comprise the polypeptide cores.

II-(i) Structure of Joint-Linker Molecular Construct

According to some embodiments of the present disclosure, the molecular construct comprises two linker units, and the linker units are coupled to each other via either the CuAAC reaction (using copper or pentamethylcyclopentadienyl ruthenium chloride complex as catalyst), the SPAAC reaction, or the iEDDA reaction. In the embodiments, one of the linker units is linked with a plurality of first elements, which act as the targeting elements, and the other of the linker units is linked with a plurality of second elements, which act as the effector elements.

According to other embodiments of the present disclosure, the molecular construct comprises three linker units, in which the first and second linker units are coupled to each other via the iEDDA reaction, and then, the third linker unit is coupled to the first or second linker unit via the CuAAC reaction. Alternatively, the first and second linker units are coupled to each other via the iEDDA reaction, and the third linker unit is coupled to the first or second linker unit via the SPAAC reaction. In the embodiments, the first, second, and third linker units respectively carry a plurality of first, second, and third elements, in which the first, second, and third elements are different. According to one embodiment, two of the three elements (i.e., the first, second, and third elements) are targeting elements, and one of the three elements is an effector element. According to another embodiment, two of the three elements are effector elements, and one of the three elements is a targeting element. According to still another embodiment, one of the three elements is a targeting element, another of the three elements is an effector element, and the other of the three elements is an element capable of improving the pharmacokinetic property of the molecular construct, such as solubility, clearance, half-life, and bioavailability.

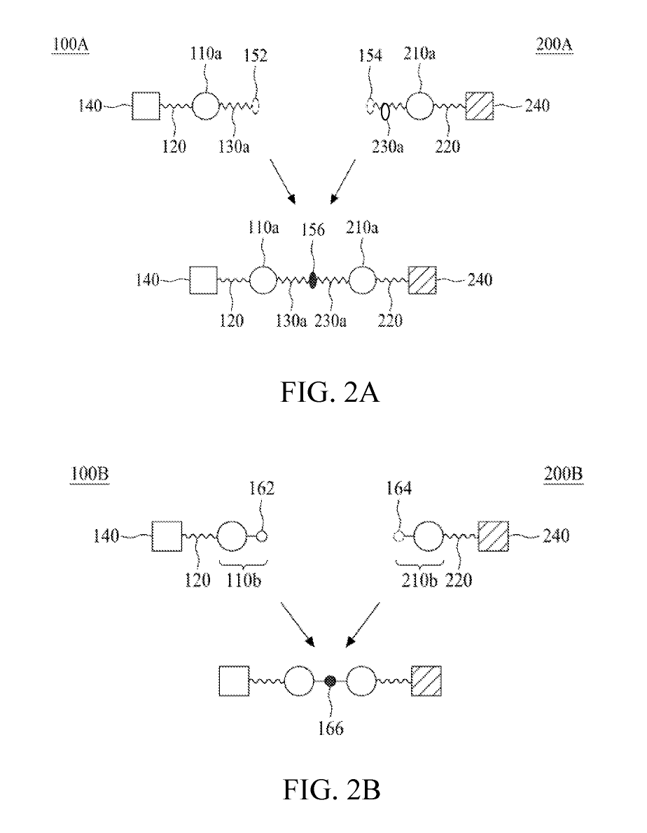

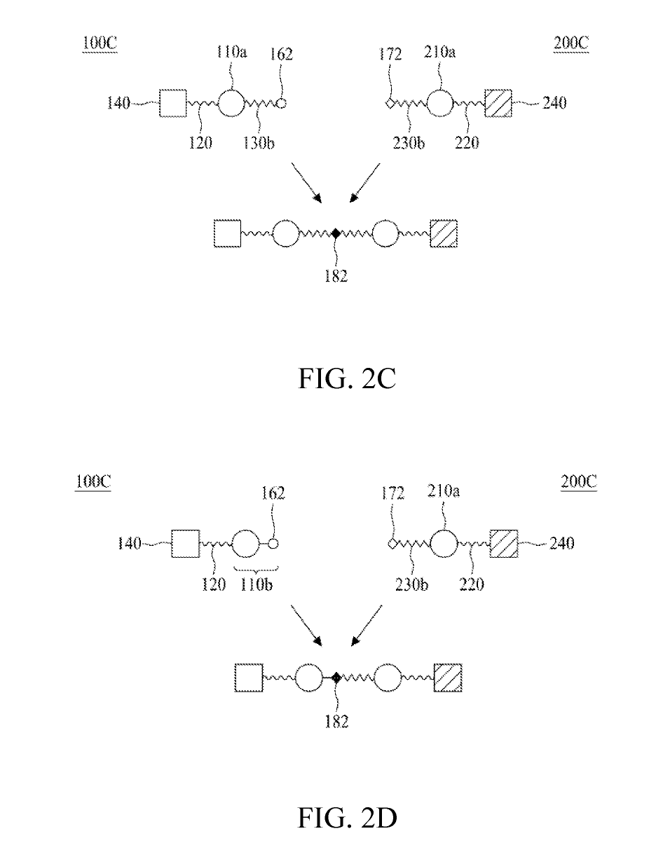

Reference is first made to FIGS. 2A-2D, which respectively depict the linkage between the two linker units. FIG. 2A depicts a molecular construct comprising two linker units (100A, 200A), which are coupled to each other via the iEDDA reaction. The first linker unit 100A comprises a first center core 110a, a linking arm 120 (as the first linking arm), and a coupling arm 130a (as the first coupling arm), in which the linking and coupling arms are respectively linked to the first center core 110a at one ends. Similarly, the second linker unit 200A comprises a second center core 210a, a linking arm 220 (as the second linking arm), and a coupling arm 230a (as the second coupling arm), in which the linking and coupling arms are respectively linked to the second center core 210a at one ends. One of the coupling arms 130a, 230a has a tetrazine group at its free terminus, while the other of the coupling arms 130a, 230a has a TCO group. Specifically, if the coupling arm 130a has a tetrazine group 152 at its free terminus (i.e., the terminus not connected to the first center core 110a), then the coupling arm 230a would have a TCO group 154 at its free terminus (i.e., the terminus not connected to the second center core 210a), and vice versa. Accordingly, the two linker units (100A, 200A) are coupled to each other via the iEDDA reaction occurred between the respective free ends of the coupling arms 130a, 230a. The ellipse 156 as depicted in FIG. 2A represents the chemical bond resulted from the iEDDA reaction occurred between the coupling arms 130a, 230a.

In the depicted embodiment, each of the linking arms 120, 220 has a maleimide group at its free terminus. Accordingly, a first targeting element 140 and a first effector element 240, each has a thiol group are respectively linked to the linking arms 120, 220 via the thiol-maleimide reaction.

FIG. 2B provides an alternative embodiment of the present disclosure, in which both the first and second center cores 110b, 210b are polypeptide cores, and are respectively linked to a first targeting element 140 and a first effector element 240 via the linking arms 120, 220. The unique feature in this embodiment is that, one of the center cores 110b, 210b comprises an amino acid residue having an azide group (e.g., the AHA residue) at it N- or C-terminus, while the other of the center cores 110b, 210b comprises an amino acid residue having an alkyne group (e.g., the HPG residue) at it N- or C-terminus, such configuration allows the center cores 110a, 210a to be directly linked to each other, that is, without connecting through any coupling arms as that depicted in FIG. 2A. Specifically, if the center core 110b comprises the amino acid residue having the azide group 162 at its N- or C-terminus, then the center core 210b would comprises the amino acid residue having the alkyne group 164 at its N- or C-terminus, and vice versa. Accordingly, the linker units 100B, 200B can couple together directly via the CuAAC reaction occurred between the N- or C-terminal amino acid residues of the center cores 110b, 210b. The solid dot 166 as depicted in FIG. 2B represents the chemical bond formed between the N- or C-terminal amino acid residues.

FIG. 2C is another embodiment of the present disclosure. The linker units 100C, 200C have the similar structures as the linker units 100A, 200A, except that the coupling arms 130b, 230b respectively have an azide group 162 and a DBCO group 172, instead of the azide group 152 and the alkyne group 154 as depicted in the linker units 100A, 200A of FIG. 2A. Specifically, the center core 110a is linked with a coupling arm 130b (as the first coupling arm) having an azide group 162 at its free-terminus; and the center core 210a is linked with a coupling arm 230b (as the second coupling arm) having a DBCO group 172 at its free-terminus. The linker units 100C, 200C are then coupled via the SPAAC reaction occurred between the coupling arms 130b, 230b; and forming the chemical bond 182, depicted as a diamond.

As would be appreciated, two linker units can be coupled to each other via the CuAAC reaction occurred between the center core and the coupling arm. Reference is now made to FIG. 2D, in which the center core 110b comprises a N- or C-terminal amino acid residue that has an azide group 162 (e.g., the AHA residue), and the center core 210a is linked with a coupling arm 230b having a TCO group 172 at its free-terminus. Accordingly, the linker units 100B and 200C can be coupled via the SPAAC reaction occurred between the center core 110b and the coupling arm 230b; and forming the chemical bond 182.

Alternatively, the linker unit that comprises a N- or C-terminal amino acid residue having an alkyne group (e.g., the HPG residue), and the linker unit comprising the coupling arm with an azide group at its free-terminus can be coupled together via the azide-alkyne cycloaddition occurred between the center core and the coupling arm.

As would be appreciated, at least one of the linker units of the present molecular construct may further comprise a connecting arm, in which one terminus of the connecting arm is linked with the linking arm, while the other terminus is linked with the functional element (either the targeting element or the effector element) as depicted in Part I. For example, the present molecular construct may comprise two linker units, in which the first element is directly linked to the first linking arm, while the second element is linked to the second linking arm via the linkage of the connecting arm. Alternatively, the present molecular construct may comprise two linker units, in which the first and second element are respectively linked to the first and second linking arms through the linkages of the first and second connecting arms.

Preferably, when at least one of the first and second linking arms is linked to the connecting arm/functional element via the CuAAC or SPAAC reaction, then the first and second linker units are coupled to each other via the iEDDA reaction. Alternatively, when at least one of the first and second linking arms is linked to the connecting arm/functional element via the iEDDA reaction, then the first and second linker units are coupled to each other via the CuAAC or SPAAC reaction.

According to some embodiments, the connecting arm is a PEG chain having 2-20 repeats of EG units. According to other embodiments, the connecting arm is a PEG chain having 2-20 repeats of EG units with a disulfide linkage at the element-linking terminus that is not linked with the linking arm.

According to one embodiment of the present disclosure, the first element is an scFv specific for transferrin receptor, and the second element is interferon-β (IFN-β), fingolimod, fingolimod phosphate, or an scFv specific for integrin α4 or β-amyloid. According to another embodiment of the present disclosure, the first element is an scFv specific for a viral protein or a bacterial protein, and the second element is an scFv specific for CD16b or CD32.

Compared with other therapeutic construct, the present molecular construct is advantageous in at least the three following aspects:

(1) the linker unit comprising a specified number and/or type of targeting/effector element can be prepared independently, then proceed to be coupled together via the CuAAC reaction, the iEDDA reaction, or the SPAAC reaction;

(2) the number and kind of the targeting and/or effector elements may vary in accordance with the requirements of application (e.g., the disease being treating, and the binding avidity and/or affinity of the targeting and/or effector element). The combination of the targeting and effector elements may be adjusted according to specific needs and/or applications. Each of the present targeting and effector elements may vary with such factors like particular condition being treated, the physical condition of the patient, and/or the type of disease being treated. The clinical practitioner may combine the most suitable targeting element and the most suitable effector element so as to achieve the best therapeutic effect. According to embodiments of the present disclosure, the targeting element may be a growth factor, a peptide hormone, a cytokine, or an antibody fragment; and the effector element may be an immunomodulant, a chelator complexed with a radioactive nuclide, a cytotoxic drug, a cytokine, a soluble receptor, or an antibody; and

(3) compared with other coupling reactions, the CuAAC reaction, the iEDDA reaction, or the SPAAC reaction is more efficient in terms of coupling any two linker units.

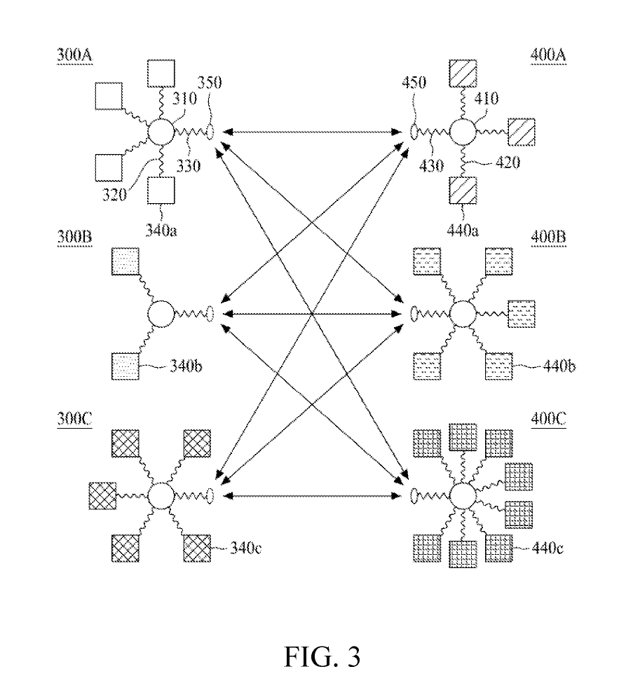

Reference is now made to FIG. 3, in which six libraries are illustrated, and are prepared independently. In this embodiment, Libraries 1-6 respectively comprise a plurality of linker units 300A, 300B, 300C, 400A, 400B, and 400C that are linked with functional elements. Each linker units 300A, 300B, and 300C are similar in structures; in which each of the linker units 300A, 300B, and 300C comprises one center core 310, one coupling arm 330 linked thereto and has a tetrazine group 350 at its free terminus, and a specified number of the linking arm 320. For instance, Linker unit 300A comprises four linking arms 320, and accordingly, four targeting elements 340a can be respectively linked to the four linking arms 320. Similarly, two targeting elements 340b and five targeting elements 340c can be respectively linked to the linker units 300B and 300C. The targeting elements 340a, 340b, and 340c can be the same or different. As to the linker units 400A, 400B and 400C, each of these linker units comprises one center core 410, one coupling arm 430 linked thereto and has a strained alkyne group 450 at its free terminus, and a specified number of the linking arm 420. As depicted, three effector elements 440a, five effector elements 440b, and eight effector elements 440c can be respectively linked to the linker units 400A, 400B and 400C. The effector elements 440a, 440b, and 440c can be the same or different. The Libraries 1-6 may be prepared independently. One skilled artisan may select the first linker unit from Libraries 1, 2 and 3, and the second linker unit from Libraries 4, 5, and 6, then proceed to couple the first and second linker units via the iEDDA reaction occurred between the tetrazine group 350 and the strained alkyne group 450 so as to produce the molecular construct with the specified number of targeting and effector elements.

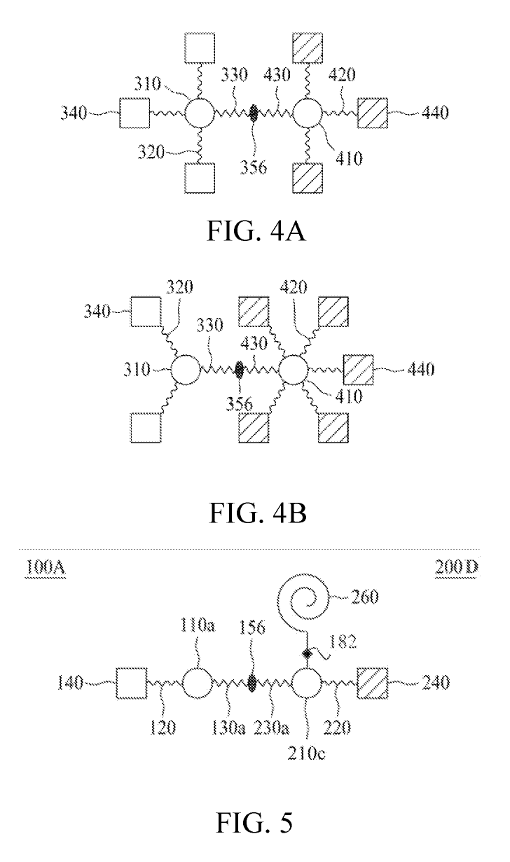

Based on the library concept, the present molecular construct can be produced with different configurations depending on the libraries selected. FIG. 4A provides an example of the present molecular construct, in which each of the first and second center cores (310, 410) is linked with three linking arms (320, 420) and one coupling arm (330, 430). Three of the first targeting elements 340 are respectively linked to the linking arms 320; and three of the first effector elements 440 are respectively linked to the linking arms 420. The two linker units are coupled to each other via the iEDDA reaction occurred between two coupling arms 330, 430, and forming the chemical bond 356. By this configuration, equal numbers of multiple targeting and/or effector elements may be carried in one molecular construct.

FIG. 4B provides another example of the present molecular construct, in which the first and second center cores respectively contain different numbers of amine groups (e.g., lysine residues), and accordingly, the molecular construct contains non-equal numbers of targeting and effector elements. In the depicted example, the first center core 310 is linked to one coupling arm 330, and two linking arms 320. The second center core 410 is linked to one coupling arm 430, and five linking arms 420. Accordingly, two targeting elements 340 are respectively linked to the linking arms 320; and five effector elements 440 are respectively linked to the linking arms 420. The ellipse 356 in FIG. 4B represents the linkage between two coupling arms 330, 430.

In optional embodiments, the present molecular construct may further comprise a relatively long PEG chain connected to either the first or second center core, so that the present molecular construct may be segregated further away from the reticuloendothelial system and attains a longer half-life after being administered to a subject. In the case where a protein is modified by a PEG chain so as to improve its pharmacokinetic properties and/or to decrease immunogenicity, PEG up to 20,000-50,000 daltons in length, is preferred. Accordingly, in one preferred embodiment of the present invention, linking arms of relatively shorter lengths are used to connect the targeting and effector elements, while a PEG chain of 20,000 to 50,000 daltons is connected to any of the linker units with the purpose of increasing in vivo half-life of the present molecular construct.

In some embodiments, multiple scFv fragments are used as the targeting and/or effector elements to construct the present molecular construct. The targeting element/effector element pharmaceuticals based on molecular constructs comprising scFv fragments should have longer in vivo half-lives than individual antibody fragments. For some clinical applications, much extended half-lives of the pharmaceuticals are desired, so as to eliminate the need of frequent administration of the drugs; in these cases, PEG chains that are 20,000 to 50,000 daltons by weight, may be used as the linking arms to link the scFv fragments that serve as targeting or effector elements. PEGs of these lengths have been used to modify a large number of therapeutic proteins to increase their half-lives.

According to some embodiments of the present disclosure, the linker unit may comprise two linking arms respectively linked to the different functional elements. Reference is now made to FIG. 5, in which the molecular construct comprises two linker units 100A and 200D. The first and second functional elements 140, 240 (one serves as the targeting element, and the other serves as the effector element) are respectively linked to the first center core 110a and the second center core 210c via the linking arms 120, 220; and the two center cores 110a, 210c are coupled to each other via the iEDDA reaction occurred between the coupling arms 130a, 230a, in which the ellipse 156 represents the chemical bond forming therebetween. In addition to the functional element 240, the second center core 210c is further linked to a PEG chain 260. Specifically, the second center core 210c comprises an AHA residue, which can be reacted with and linked to the PEG chain 260 having a stained alkyne group via the SPAAC reaction, in which the diamond 182 represents the chemical bond forming from the SPAAC reaction. Depending on the intended and desired use, the third element can be a second targeting element, a second effector element, or an element capable of improving the pharmaceutical property of the molecular construct. According to one embodiment of the present disclosure, the PEG chain 260 has a molecular weight about 20,000 to 50,000 daltons.

Based on the concept, a linker unit may comprise a plurality of linking arms, which can be linked to a plurality of functional elements. For example, a linker unit may comprises 5-12 linking arms, which can be linked to 5-12 functional elements. This is especially useful when the functional elements are small molecules, such as therapeutic drugs or toll-like receptor agonists. The linker unit carrying multiple molecules of a therapeutic drug is herein referred to as a drug bundle.

Further, the polypeptide cores can be employed to prepare the molecular construct comprising three linker units. Accordingly, another aspect of the present disclosure is directed to a molecular construct comprising three linker units. Among the three linker units, two of them may be connected to each other via the iEDDA reaction, while the third linker unit is connected to any of the two linker units by the SPAAC reaction or CuAAC reaction. The rationale for constructing a multi-linker unit (e.g., three linker units) is that two different sets of targeting elements or two different sets of effector elements can be incorporated therein.

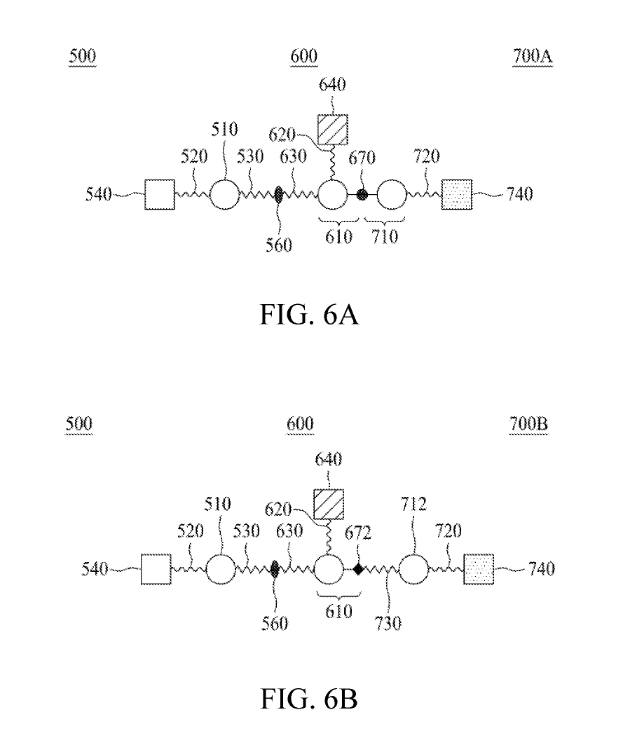

Reference is now made to FIG. 6A, in which the molecular construct comprises three linker units (500, 600, 700A). The linker units 500, 600, 700A respectively comprise a center core (510, 610, 710), and a linking arm (520, 620, 720) with a functional element (540, 640, 740) linked thereto. The linker unit 600 is characterized in comprising a cysteine residue at one of its N- or C-terminus that is linked with a coupling arm 630; and an amino acid residue having an azide or alkyne group at the other of its N- or C-terminus. One of the coupling arms 530, 630 has a tetrazine group at its free terminus, and the other of the coupling arms 530, 630 has a strained alkyne group at its free terminus. Accordingly, the linker units 500, 600 can be coupled to each other via the iEDDA reaction occurred between the coupling arms 530, 630 as the linkage manner described in FIG. 2A. As to the linkage of the linker unit 700, when the N- or C-terminal amino acid residue of the center core 610 has an azide group (e.g., the AHA residue), the center core 710 comprises an amino acid having an alkyne group (e.g., the HPG residue) at its N- or C-terminus; or, when the N- or C-terminal amino acid residue of the center core 610 has an alkyne group (e.g., the HPG residue), then the center core 710 comprises an amino acid having an azide group (e.g., the AHA residue) at its N- or C-terminus. Thus, as the linkage manner described in FIG. 2B, the linker units 600, 700A can be directly coupled to each other via the CuAAC reaction occurred between the N- or C-terminal amino acid residues of the center cores 610, 710 without the presence of the coupling arms. The ellipse 560 and the solid dot 670 in FIG. 6A respectively represent the chemical bonds resulted from the iEDDA reaction and the CuAAC reaction.

Alternatively, two of the three linker units may be connected to each other via the iEDDA reaction, while the third linker unit is connected to any of the two linker units by the SPAAC reaction. Reference is now made to FIG. 6B, in which the linker units 500, 600 are coupled together via the iEDDA reaction as described in FIG. 6A, whereas the linker unit 700B is linked to the linker unit 600 via the SPAAC reaction occurred between the center core 610 and the coupling arm 730. The diamond 672 in FIG. 6B represents the chemical bond resulted from the SPAAC reaction.

As would be appreciated, each number of the functional elements 540, 640, 740 respectively linked to the linker units 500, 600, 700A or 700B are different depending on the intended use. With the library concept depicted in FIG. 3, the linker units respectively carrying different numbers and/or types of functional elements can be prepared separately as different libraries, and one skilled artisan may select and combine the desired linker units from the libraries in accordance with the various applications.

Basically, the coupling arm of the present molecular construct described in above aspects and/or embodiments of the present disclosure that has an azide, alkyne, tetrazine, or strained alkyne group at the terminus is designed as a PEG chain having 2-12 repeats of EG units. The linking arm is designed as a PEG chain having 2-20 repeats of EG units; preferably, the linking arm is a PEG chain having 2-20 repeats of EG units with a disulfide linkage at the free terminus that is not linked with the center core.

Adopting a polypeptide as the center core provides versatility in the present molecular construct, in which multiple copies or types of targeting/effector elements may be present in one construct, accordingly, enhanced specificity of drug delivery and potency in the intended target sites are achieved. A large number of configurations can be adopted by employing the molecular construct comprising multiple linker units. A few examples are: a first linker unit carrying three scFvs targeting elements, and a second linker unit carrying 5 therapeutic drugs; a first linker unit carrying three scFvs targeting elements, and a second linker unit carrying three scFvs effector elements; a first linker unit carrying two scFvs of the first set targeting elements, a second linker unit carrying two scFvs of the second set targeting elements, and a third linker unit carrying 5 therapeutic drugs; a first linker unit carrying 2 bi-scFv targeting elements, and a second linker unit carrying two scFvs effector elements; or a first linker unit carrying three scFvs targeting elements, a second linker unit carrying two scFvs effector elements plus a linking arm attached with a long PEG of 20,000-50,000 daltons for the purpose of increasing pharmacokinetic properties.

In some embodiments of this invention, a bi-functional PEG acting as a linking arm is used to link the antigen-binding fragments of antibodies, which serve as targeting or effector elements, to the amine groups located in the polypeptide core. Each PEG may have NHS group at one end and maleimide group at the other end. The NHS group may couple with amine group in the polypeptide core, while the maleimide group may couple with sulfhydryl group of a cysteine residue of an scFv, bi-scFv, or Fab fragment of an antibody. The scFv and bi-scFv are engineered to have a polypeptide linker with terminal cysteine residue at the C-terminal. Fab may be derived from a whole IgG by pepsin cleavage, and the free sulfhydryl groups are derived from the inter-chain disulfide bond by a mild reduction reaction.

When the targeting and effector elements are all scFv, and linking arms of 600 daltons (12 EG units) are used, a molecular construct with a total of six scFvs has a molecular weight of about 170,000 daltons. A molecular construct with seven scFvs has a molecular weight of about 200,000 daltons, and a molecular construct with eight scFvs has a molecular weight of about 230,000 daltons. Most of the molecular constructs of this invention have molecular weights smaller than 200,000 daltons, and a few molecular constructs have molecular weights in 200,000-250,000 daltons.

When four different sets of scFv are to be carried in one molecular construct, it is preferable to have one linker unit carrying a joined single-chain, bi-specific scFv (bi-scFv), such as scFv1-scFv2 (e.g., specific for HER2 and HER3), and the other two linker units each carrying one scFv (i.e., scFv3 and scFv4 respectively). There are two ways to construct bi-specific scFv1-scFv2. In the “tandem” configuration, V_{L}1-V_{H}1-V_{L}2-V_{H}2 or V_{H}1-V_{L}1-V_{H}2-V_{L}2 is arranged; in the “diabody” configuration, V_{L}2-V_{L}1-V_{H}1-V_{H}2 or V_{H}2-V_{H}1-V_{L}1-V_{L}2 is arranged. Proper linkers with GGGGS (SEQ ID NO: 26) repeats or other sequences are placed between the immunoglobulin domains.

In our experience, a peptide or a PEG linker, which contain maleimide and azide groups may become polymerized upon long-term storage, due to the automatic coupling reaction between the maleimide and azide groups. Therefore, it is preferable that each linker unit is prepared freshly and independently, and processed to connecting the targeting or effector elements onto the linker units, and the coupling of the linker units through click reaction without delay. An alternative preferred embodiment is that the targeting elements and effector elements are both conjugated to linker units with alkyne groups, and the alkyne group in one of the linker units is then converted to azide with a short homo-bifunctional linker with azide at both ends. The linker units, one with alkyne and the other with azide, are then coupled via a click reaction.

The preferred linking arms for this invention are PEG. The length of the linking arms is important for several considerations. It should be long enough to allow flexibility of the linked scFv or other types of functional elements to reach targeted antigenic sites on targeted cell surface without steric constraints; yet not long enough to cause intra-molecular and inter-molecular tangling of the linking arms and their linked scFv fragments or functional elements, or to unnecessarily increase the size of the whole molecular construct for hindering tissue penetration. Linking arms that are too long may also fail to pull antigen molecules to form compacted clusters, if such clusters are required to initiate signal-transducing process for apoptosis or other cellular effects. The optimal length of linking arms for different types of combinations of targeted antigens and their binding agents may be determined by any skilled artisan in the related field without undue experimentation. A linking arm of NHS-(PEG)₁₂-Maleimide (approximately 500 daltons) is preferred in a number of molecular construct of this invention. A fully stretched (PEG)₁₂ has a length of 40-50 Å.

Applicable linking arms, coupling arms and connecting arms are not limited by PEG chains. Peptides comprising glycine, serine and other amino acid hydrophilic residues, and polysaccharides, and other biocompatible linear polymers, which are modified to contain functional groups (e.g., an NHS, a maleimide, an azide, an alkyne, a tetrazine, or a strained alkyne group), can be used.

For certain therapeutic applications, it is desirable that the effector elements in the molecular constructs of this disclosure be released from the linking arms, so that they can get into cells in the targeted site, including cells bound by the targeting elements or surrounding cells, to cause pharmacological effects. In those cases, a cleavable bond is engineered in the linking arm. Cleavable bonds, which are susceptible for cleavage by hydrolysis, acid exposure, reduction, and enzymes, have been developed. For example, peptide segments susceptible to matrix metalloproteinases, which are present in inflammatory tissues, have been used in constructing therapeutic constructs. One embodiment of the present invention is to use PEG linkers with S—S bond adjacent to the maleimide group NHS-PEG₂₋₁₂-S—S-maleimide, wherein S—S is a disulfide bond, which can be slowly reduced.

According to some embodiments of the present disclosure, the targeting element described in above-mentioned embodiments is selected from the group consisting of a growth factor, a peptide hormone, a cytokine, and an antibody fragment; and the effector element is an immunomodulant, a chelator complexed with a radioactive nuclide, a therapeutic drug, a cytokine, a soluble receptor, or an antibody.

In the embodiments, the antibody is in the form of an antigen-binding fragment (Fab), a variable fragment (Fv), a single-chain variable fragment (scFv), a single domain antibody (sdAb), or a bi-specific single-chain variable fragment (bi-scFv). According to one embodiment, the bi-scFv is a bi-specific tandem scFv or a bi-specific diabody scFv.

In order to retain diffusing ability of the molecular constructs, a molecular size smaller than 250,000 daltons is preferred. Thus, scFv fragments are preferred for most of the embodiments. At the DNA level, genes are constructed so that the V_{L} and V_{H} are linked as a single polypeptide in either order (V_{L}-V_{H} or V_{H}-V_{L}) by a peptide linker of 10-25 amino acid residues with glycine and serine being the major residues. At the C-terminal, a short stretch with glycine and serine and a terminal residue cysteine is engineered. Recombinant scFv and bi-scFv can be produced in bacteria, such as E. coli and Pseudomonas putida, in yeast, such as Pichia pastoris, or in mammalian cells, such as CHO and HEK293 cell lines.

The inventors' laboratory have produced a large number of IgG antibodies, Fab, scFv and various antibody fragments, Fc-based proteins, and other recombinant antibodies in HEK293 and CHO cell lines for experimentation in in vitro systems and in animal models. Our laboratory has also developed cell lines for producing antibodies for human clinical trials. The HEK293 transient expression system can be conveniently employed to produce up to 1 g of IgG or antibody fragments using a few flasks of 1-2 liters in the research laboratory. The scFv fragments to be used in the molecular constructs of this invention generally do not have a carbohydrate modification, and carbohydrate modification is not required for the binding activity of the scFv to their antigenic targets. Furthermore, only one disulfide bond and one terminal cysteine are present in the scFv fragment. Therefore, small-scale bacterial expression systems have been developed as a manufacturing alternative for producing scFv. With E. coli, expression systems for recovering scFv in intracellular inclusion bodies, in periplasm, and in secreted form have been employed. The scFv can be purified in most cases with an affinity column with Protein L, which interacts with V_{H} of most κ light chain, or in other cases with ion-exchange columns.

The examples of this invention based on the joint-linker platform employ mainly scFv and Fab as the targeting and/or effector elements. However, specific binding molecules may also be screened from large libraries of binding molecules based on sdAb or other antibody fragments. Libraries of binding molecules, which are not based on immunoglobulin domains but resemble antibodies in having specific binding affinities to selected target molecules, include (1) aptamers, which are oligonucleotides or short peptides selected for binding to target molecules, (2) fynomers, which are small binding proteins derived from the human Fyn SH3 domain, (3) affimers, which are binding proteins derived from the cysteine protein inhibitor family of cystatins, and (4) DARPins (designed ankyrin repeat proteins), which are genetically engineered proteins with structures derived from the natural ankyrin proteins and consist of 3, 4, or 5 repeat motifs of these proteins. These antibody-mimetics have molecular weights of about 10K to 20K daltons.

II-(ii) Functional Elements Suitable for Use with Joint-Linker Molecular Construct

As discussed above, the present joint-linker comprises at least two linker units, in which the first linker unit carries one or more targeting elements, and the second linker unit carries one or more effector elements or pharmacokinetic property-enhancing elements, and vice versa. The skilled artisan may select suitable functional elements as the targeting element, effector element and/or pharmacokinetic property-enhancing element in accordance with the first and second elements selected in Part I-(ii) of this specification so as to produce the desired effect.

II-(iii) Use of Joint-Linker Molecular Construct

The present disclosure also pertains to method for treating various diseases using the suitable joint-linker molecular construct, including an immune disorder, a diffused tumor, a solid tumor, an osteoporosis disease, an AMD, a CNS disease, an infectious disease, blood clot-related disease (e.g., thrombosis) and transplantation rejection. Generally, the method comprises the step of administering to a subject in need of such treatment an effective amount of the joint-linker molecular construct according to embodiments of the present disclosure.

### EXAMPLES

### Example 1

### Synthesis of Peptide 1, 2 and 3 (SEQ ID NO: 37, 38 and 39) as Peptide Cores for Constructing Multi-Arm Linker Units

Peptides 1 to 3 were synthesized by Shanghai ChinaPeptide Co., Ltd. (Shanghai, China) using a standard solid phase method, and the purity of peptide 1 to 3 were respectively 95.86%, 95.64%, and 97.49%. Each of the synthesized peptides was used as a central core for constructing multi-arm linker units.

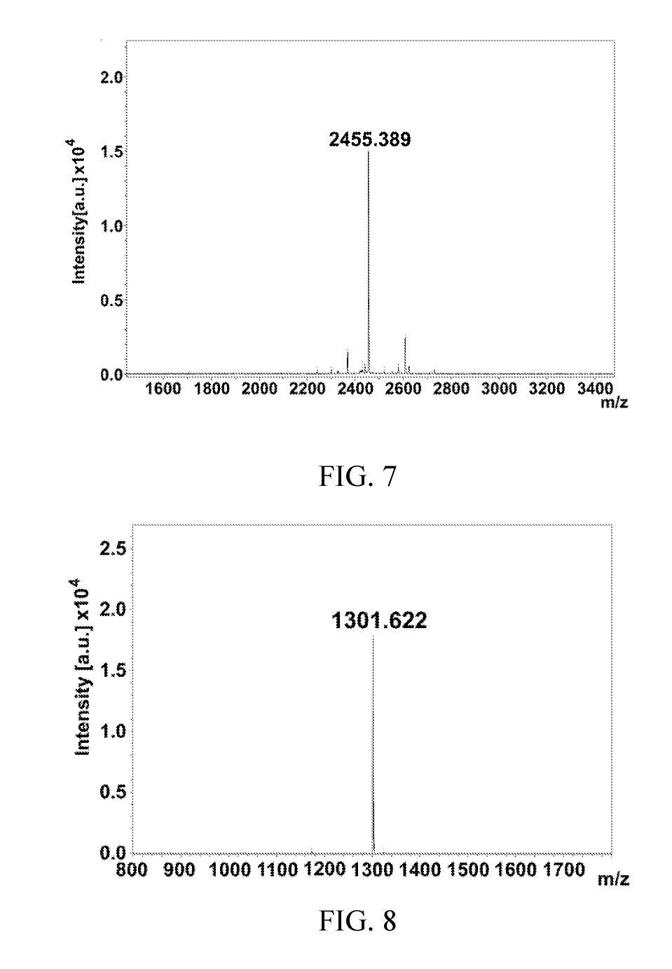

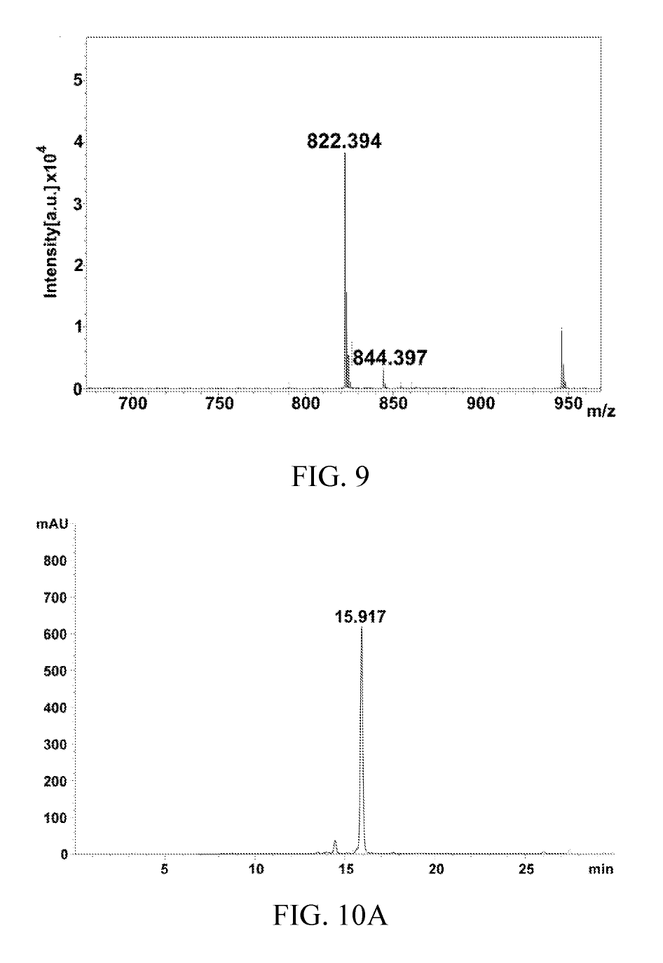

The synthesized peptides were respectively identified by MALDI-TOF mass spectrometry (Bruker Autoflex III MALDI-TOF/TOF mass spectrometer, Bruker Daltonics, Bremen, Germany) performed. Results are provided in FIGS. 7 to 9. As illustrated in the relevant figures, peptides 1, 2, and 3 (SEQ ID NO: 37, 38 and 39) respectively had a molecular weight of 2455.39, 1301.62, and 822.39 daltons.

### Example 2

### Conjugating SH Group of C Residue of Peptide 3 (SEQ ID NO: 39) with Maleimide-PEG₃-Methyltetrazine of a Coupling Arm

Peptide 3 (ChinaPeptide Co., Ltd.) was dissolved in 100% DMSO at a final concentration of 10 mM. For conjugating the SH group of C residue with maleimide-PEG₃-methyltetrazine (Click Chemistry Tools Inc., Scottsdale. USA) to create a functional linking group methyltetrazine, the peptide and maleimide-PEG₃-methyltetrazine were mixed at a 1:1 ratio and incubated at room temperature over 18 hours. Methyltetrazine-conjugated peptide was purified by reverse phase HPLC on a PrincetonSPHER-300 C18 column (250 mm×30 mm; 300 Å; 5 μm), using a mobile phase of acetonitrile and 0.1% trifluoroacetic acid, a linear gradient of 0% to 73% acetonitrile over 28 minutes, at a flow rate of 27 mL/min and a column temperature of 25° C.

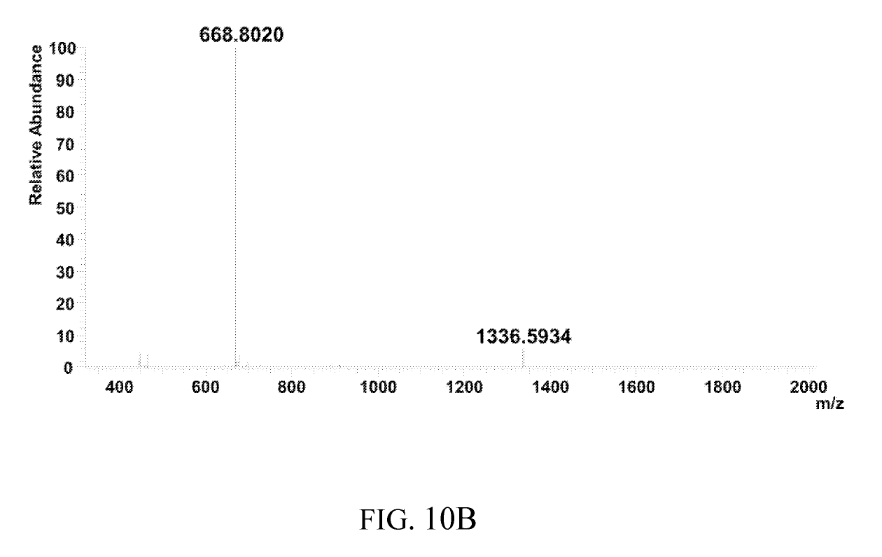

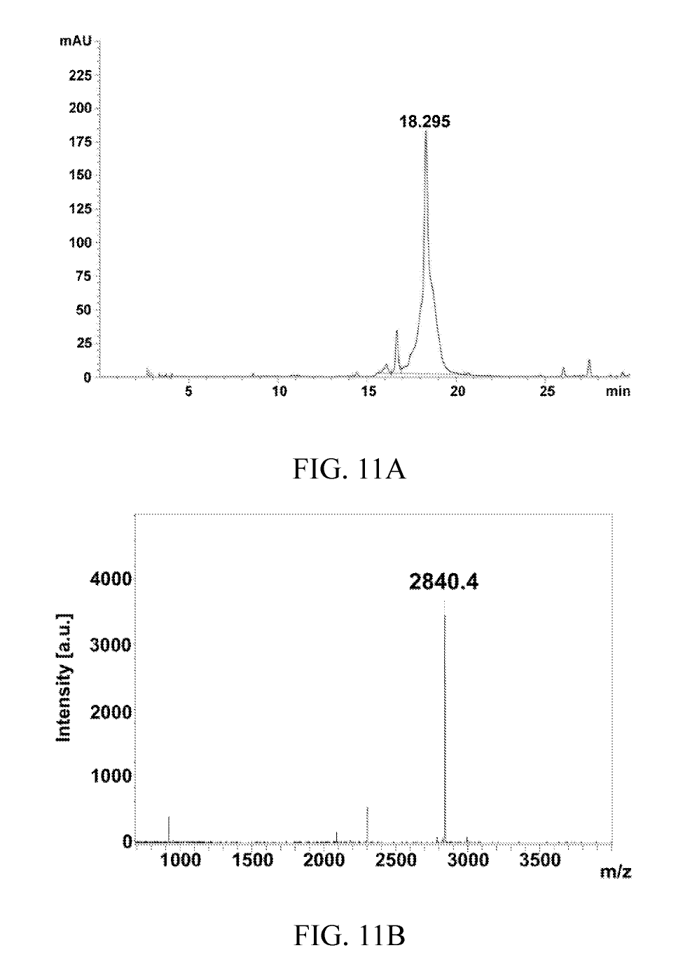

The purified sample of methyltetrazine-peptide 3 was analyzed by reverse phase analytical HPLC on a Macherey-Nagel MN Nucleodur C18 Pyramid column (250 mm×4.6 mm; 5 μm), using a mobile phase of acetonitrile and 0.1% trifluoroacetic acid, a linear gradient of 0% to 75% acetonitrile over 22 minutes, at a flow rate of 1.0 ml/min and a column temperature of 25° C.; The result as illustrated in FIG. 10A indicated that methyltetrazine-peptide 3 had a retention time of 15.917 minutes.

The synthesized methyltetrazine-peptide 3 as depicted below was identified by mass spectrometry ESI-MS using a LTQ Orbitrap XL ETD mass spectrometer (Thermo Fisher Scientific, San Jose, Calif.) equipped with standard ESI ion source. The two peaks as illustrated in FIG. 10B respectively corresponded to [M+H]⁺ of methyltetrazine-peptide 3, which had a molecular weight of 1336.59 daltons; and [M+2H]²⁺ of peptide 3, which had a molecular weight of 668.8 daltons.

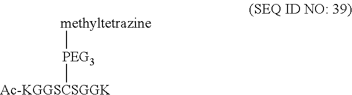

### Example 3

### Synthesis of a Multi-Arm Linker Unit By Conjugating NHS-PEG₁₂-Mal to —NH₂ Groups of Methyltetrazine-Peptide 3

In this examples, two linking arms of PEG₁₂-maleimide were conjugated to the peptide core methyltetrazine-peptide 3 of Example 2. The crosslinker, NHS-PEG₁₂-maleimide (succinimidyl-[(N-maleimido-propionamido)-dodecaethyleneglycol] ester, was purchased from Conju-probe Inc. The conjugation was performed in accordance with the manufacturer's instruction. The peptide was dissolved in 100% DMSO at a final concentration of 10 mM. NHS-PEG₁₂-maleimide crosslinker was added to the dissolved peptide at a final concentration of 60 mM (6-fold molar excess over 10 mM peptide solution). Organic base DABCO (1,4-diazabicyclo[2.2.2]octane) (5 equiv) was added to the reaction mixtures serving as a catalyst. The reaction mixtures were incubated over 18 hours at room temperature. The maleimide-PEG₁₂-conjugated methyltetrazine-peptide 3 was purified by reverse phase HPLC on a Princeton SPHER-300 C18 column (250 mm×30 mm; 300 Å; 5 μm), using a mobile phase of acetonitrile and 0.1% trifluoroacetic acid, a linear gradient of 0% to 73% acetonitrile over 28 minutes, at a flow rate of 27.0 ml/min and a column temperature of 25° C.

The purified sample of maleimide-PEG₁₂-conjugated methyltetrazine-peptide 3 was then analyzed by reverse phase analytical HPLC on a Macherey-Nagel MN Nucleodur C18 Pyramid column (250 mm×4.6 mm; 5 μm), using a mobile phase of acetonitrile and 0.1% trifluoroacetic acid, a linear gradient of 0% to 75% acetonitrile over 22 minutes, at a flow rate of 1.0 ml/min and a column temperature of 25° C. The data of FIG. 11A indicated the maleimide-PEG₁₂-conjugated methyltetrazine-peptide 3 had a retention time of 18.295 minutes.

As illustrated below, the thus-synthesized maleimide-PEG₁₂-conjugated methyltetrazine-peptide 3 had one coupling arm with a methyltetrazine group, and two PEG linking arms respectively having maleimide groups; and a molecular weight of 2840.4 daltons (FIG. 11B).

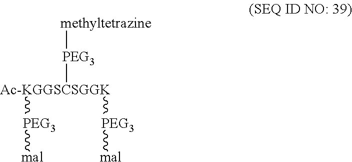

What is claimed is:

1. A linker unit comprising a center core, a plurality of linking arms, and optionally a coupling arm having an azide, an alkyne, a tetrazine, a cyclooctene, or a cyclooctyne group at its free terminus, wherein
the center core comprises,
(1) 2 to 15 lysine (K) residues;
(2) one or more conjugating sequences, disposed at the N- or C-terminus of the center core or between two consecutive K residues of the 2 to 15 K residues, wherein each of the conjugating sequences independently comprises a conjugating amino acid residue that is a cysteine (C) residue or an amino acid residue having an azide or an alkyne group, wherein when the conjugating amino acid residue is the C residue, then the thiol group of the C residue is linked with the coupling arm; and
(3) optionally, one or more filler sequences, disposed between two consecutive K residues of the 2 to 15 K residues, wherein each of the filler sequences independently comprises two or more amino acid residues other than the conjugating amino acid residue, and at least one of the filler sequences is devoid of glycine (G), serine (S), or a combination thereof;
the plurality of linking arms are respectively linked to the 2 to 15 K residues of the center core, wherein each of the plurality of linking arms has a N-hydroxysuccinimidyl (NHS), the azide, the alkyne, the tetrazine, the cyclooctene, or the cyclooctyne group at its free terminus; and
when the free terminus of the linking arm is the azide, the alkyne, or the cyclooctyne group, then the conjugating amino acid residue is the C residue, and the free terminus of the coupling arm is the tetrazine or the cyclooctene group; or
when the free terminus of the linking arm is the tetrazine group or cyclooctene group, then the conjugating amino acid residue is the C residue or the amino acid residue having the azide or the alkyne group and the free terminus of the coupling arm is the azide, the alkyne, or the cyclooctyne group.

2. The linker unit of claim 1, wherein each of the conjugating sequence is disposed between two consecutive K residues of the 2 to 15 K residues.

3. The linker unit of claim 1, wherein
each of the linking arms is a PEG chain having 2-20 repeats of EG units or a PEG chain having 2-20 repeats of EG units with a disulfide linkage at the free terminus thereof; and
the coupling arm is a PEG chain having 2-12 repeats of EG units.

4. The linker unit of claim 1, wherein
the amino acid residue having the azide group is L-azidohomoalanine (AHA), 4-azido-L-phenylalanine, 4-azido-D-phenylalanine, 3-azido-L-alanine, 3-azido-D-alanine, 4-azido-L-homoalanine, 4-azido-D-homoalanine, 5-azido-L-ornithine, 5-azido-d-ornithine, 6-azido-L-lysine, or 6-azido-D-lysine;
the amino acid residue having the alkyne group is L-homopropargylglycine (L-HPG), D-homopropargylglycine (D-HPG), or beta-homopropargylglycine (β-HPG);
the cyclooctene group is trans-cyclooctene (TCO); and the cyclooctyne group is dibenzocyclooctyne (DBCO), difluorinated cyclooctyne(DIFO), bicyclononyne (BCN), or dibenzocyclooctyne (DICO); and
the tetrazine group is 1,2,3,4-tetrazine, 1,2,3,5-tetrazine or 1,2,4,5-tetrazine, or derivatives thereof.

5. The linker unit of claim 1, further comprising a plurality of first elements that are respectively linked to the plurality of linking arms via forming an amide bound therebetween, or via copper catalyzed azide-alkyne cycloaddition (CuAAC) reaction, strained-promoted azide-alkyne click chemistry (SPAAC) reaction, or inverse electron demand Diels-Alder (iEDDA) reaction.

6. The linker unit of claim 5, further comprising a second element that is linked to the center core via any of the following reactions,
CuAAC reaction occurred between the azide or the alkyne group and the second element;
SPAAC reaction occurred between the azide or cyclooctyne group and the second element; and
iEDDA reaction occurred between the cyclooctene group or tetrazine group and the second element.

7. The linker unit of claim 6, wherein the center core comprises two conjugating sequences, wherein
one of the conjugating sequences comprises the amino acid residue having the azide or alkyne group, and
the other of the conjugating sequences comprises the C residue.

8. The linker unit of claim 6, further comprising a third element, wherein
the plurality of first elements are respectively linked to the plurality of linking arms via forming the amide bound therebetween,
the second element is linked to the azide or alkyne group via CuAAC or SPAAC reaction, and
the third element is linked to the coupling arm linked with the C residue via iEDDA reaction.

9. The linker unit of claim 1, further comprising a plurality of connecting arms that are respectively linked to the plurality of linking arms via CuAAC reaction, SPAAC reaction, or iEDDA reaction, wherein each of the plurality of connecting arms has a maleimide or the NHS group at its free terminus.

10. The linker unit of claim 9, wherein each of the connecting arms is a PEG chain having 2-20 repeats of EG units or is a PEG chain having 2-20 repeats of EG units with a disulfide linkage at the terminus that is not linked with the linking arm.

11. The linker unit of claim 9, further comprising a plurality of first elements that are respectively linked to the plurality of linking arms via thiol-maleimide reaction or forming an amide bound therebetween.

12. The linker unit of claim 11, further comprising a second element that is linked to the center core via any of the following reactions:
CuAAC reaction occurred between the azide or the alkyne group and the second element;
SPAAC reaction occurred between the azide or cyclooctyne group and the second element; and
iEDDA reaction occurred between the cyclooctene group or tetrazine group and the second element.

13. A molecular construct comprising a first linker unit and a second linker unit, wherein
the first linker unit comprises,
a first center core,
a first linking arm linked to the first center core,
optionally, a first connecting arm linked to the first linking arm,
a first element linked to the first linking arm or the first connecting arm, and
optionally, a first coupling arm linked to the first center core;
the second linker unit comprises,
a second center core,
a second linking arm linked to the second center core,
optionally, a second connecting arm linked to the second linking arm,
a second element linked to the second linking arm or the second connecting arm, and
optionally, a second coupling arm linked to the second center core; and
the first and second linker units are coupled to each other via CuAAC reaction, SPAAC reaction or iEDDA reaction occurred between any of the followings: the first and second center cores, the first coupling arm and the second center core, the first and second coupling arms, or the first center core and the second coupling arm.

14. The molecular construct of claim 13, further comprising a first and a second elements respectively linked to the first and second linking arms.

15. The molecular construct of claim 13, further comprising a first and a second connecting arms respectively linked to the first and second linking arms.

16. The molecular construct of claim 15, further comprising a first and a second elements respectively linked to the first and second connecting arms.

17. The molecular construct of claim 13, wherein,
each of the first and second linking arms is a PEG chain having 2-20 repeats of EG units or a PEG chain having 2-20 repeats of EG units with a disulfide linkage at the free terminus thereof; and
each of the first and second coupling arms is a PEG chain having 2-12 repeats of EG units.

18. The molecular construct of claim 13, wherein each of the first and second connecting arms is the PEG chain having 2-20 repeats of EG units or the PEG chain having 2-20 repeats of EG units with a disulfide linkage at the terminus that is not linked with the linking arm.

19. The molecular construct of claim 13, wherein,
one of the first and second coupling arms has an azide group at the free-terminus thereof, and the other of the first and second coupling arms has an alkyne or a cyclooctyne group at the free-terminus thereof; and
the first and second linker units are coupled to each other via CuAAC reaction or SPAAC reaction occurred between the first and second coupling arms.

20. The molecular construct of claim 13, wherein,
one of the first and second coupling arms has a tetrazine group at the free-terminus thereof, and the other of the first and second coupling arms has a cyclooctene group at the free-terminus thereof; and
the first and second linker units are coupled to each other via iEDDA reaction occurred between the first and second coupling arms.


SEQUENCE LISTING (40 sequences)

SEQ ID NO: 1 - protein, 4 aa, Artificial Sequence
  artificial filler sequence-1
SRRS

SEQ ID NO: 2 - protein, 4 aa, Artificial Sequence
  artificial filler sequence-2
SRHS

SEQ ID NO: 3 - protein, 4 aa, Artificial Sequence
  artificial filler sequence-3
SHHS

SEQ ID NO: 4 - protein, 4 aa, Artificial Sequence
  artificial filler sequence-4
SEES

SEQ ID NO: 5 - protein, 5 aa, Artificial Sequence
  artificial filler sequence-5
SRSRS

SEQ ID NO: 6 - protein, 5 aa, Artificial Sequence
  artificial filler sequence-6
SHSHS

SEQ ID NO: 7 - protein, 5 aa, Artificial Sequence
  artificial filler sequence-7
SRSHS

SEQ ID NO: 8 - protein, 5 aa, Artificial Sequence
  artificial filler sequence-8
SDSDS

SEQ ID NO: 9 - protein, 5 aa, Artificial Sequence
  artificial filler sequence-9
SESES

SEQ ID NO: 10 - protein, 5 aa, Artificial Sequence
  artificial filler sequence-10
SRRRS

SEQ ID NO: 11 - protein, 5 aa, Artificial Sequence
  artificial filler sequence-11
SHHHS

SEQ ID NO: 12 - protein, 5 aa, Artificial Sequence
  artificial filler sequence-12
SRHRS

SEQ ID NO: 13 - protein, 5 aa, Artificial Sequence
  artificial filler sequence-13
SDDDS

SEQ ID NO: 14 - protein, 5 aa, Artificial Sequence
  artificial filler sequence-14
SEEES

SEQ ID NO: 15 - protein, 5 aa, Artificial Sequence
  artificial filler sequence-15
SRDRS

SEQ ID NO: 16 - protein, 6 aa, Artificial Sequence
  artificial filler sequence-16
SRSSRS

SEQ ID NO: 17 - protein, 6 aa, Artificial Sequence
  artificial filler sequence-17
SRHHRS

SEQ ID NO: 18 - protein, 6 aa, Artificial Sequence
  artificial filler sequence-18
SHRRHS

SEQ ID NO: 19 - protein, 6 aa, Artificial Sequence
  artificial filler sequence-19
SSDDSS

SEQ ID NO: 20 - protein, 6 aa, Artificial Sequence
  artificial filler sequence-20
SRDDRS

SEQ ID NO: 21 - protein, 4 aa, Artificial Sequence
  artificial filler sequence-21
GGGS

SEQ ID NO: 22 - protein, 4 aa, Artificial Sequence
  artificial filler sequence-22
GSGS

SEQ ID NO: 23 - protein, 4 aa, Artificial Sequence
  artificial filler sequence-23
GGSG

SEQ ID NO: 24 - protein, 5 aa, Artificial Sequence
  artificial filler sequence-24
GSGGS

SEQ ID NO: 25 - protein, 5 aa, Artificial Sequence
  artificial filler sequence-25
SGGSG

SEQ ID NO: 26 - protein, 5 aa, Artificial Sequence
  artificial filler sequence-26
GGGGS

SEQ ID NO: 27 - protein, 6 aa, Artificial Sequence
  artificial filler sequence-27
GGSGGS

SEQ ID NO: 28 - protein, 7 aa, Artificial Sequence
  artificial filler sequence-28
GGSGGSG

SEQ ID NO: 29 - protein, 8 aa, Artificial Sequence
  artificial filler sequence-29
SGSGGSGS

SEQ ID NO: 30 - protein, 9 aa, Artificial Sequence
  artificial filler sequence-30
GSGGSGSGS

SEQ ID NO: 31 - protein, 10 aa, Artificial Sequence
  artificial filler sequence-31
SGGSGGSGSG

SEQ ID NO: 32 - protein, 11 aa, Artificial Sequence
  artificial filler sequence-32
GGSGGSGGSGS

SEQ ID NO: 33 - protein, 12 aa, Artificial Sequence
  artificial filler sequence-33
SGGSGGSGSGGS

SEQ ID NO: 34 - protein, 13 aa, Artificial Sequence
  artificial filler sequence-34
GGGGSGGSGGGGS

SEQ ID NO: 35 - protein, 14 aa, Artificial Sequence
  artificial filler sequence-35
GGGSGSGSGSGGGS

SEQ ID NO: 36 - protein, 15 aa, Artificial Sequence
  artificial filler sequence-36
SGSGGGGGSGGSGSG

SEQ ID NO: 37 - protein, 21 aa, Artificial Sequence
  MOD_RES 1: ACETYLATION
  synthesized peptide 1
CSRSKSRSKSRSKSRSKSRSK

SEQ ID NO: 38 - protein, 13 aa, Artificial Sequence
  synthesized peptide 2
  MOD_RES 9: Xaa is homopropargylglycine
KSSSKGEGXGEGK

SEQ ID NO: 39 - protein, 9 aa, Artificial Sequence
  MOD_RES 1: ACETYLATION
  synthesized peptide 3
KGGSCSGGK

SEQ ID NO: 40 - protein, 11 aa, Artificial Sequence
  artificial sequence
  MOD_RES 3: Xaa is homopropargylglycine
GSXGGKGGSSK